(19) United States
(12) Patent Application Publication
(10) Pub. No.: US 2007/0031511 A1
(43) Pub. Date: Feb. 8, 2007

(54) Method of treatment

(76) Inventors: Graham S Baldwin, Pascoe Vale South (AU); Kevin Jeffrey Barnham, Coburg (AU); Julie Pannequin, Paris (FR); John-Paul Tantiongco, Bundoora (AU); Arthur Shulkes, Ascot Vale (AU); Raymond Stanley Norton, Ivanhoe (AU); Suzana Kovak, Aspendale (AU); Hong He, Kensington (AU); Brian Philip Sheehan, Brunswick (AU)

Correspondence Address: NEEDLE & ROSENBERG, P.C., SUITE 1000, 999 PEACHTREE STREET, ATLANTA, GA, 30309-3915, US

(21) Appl. No.: 10/552,381

(22) PCT Filed: Apr. 8, 2004
(86) PCT No.: PCT/AU04/00474

§ 371 Date: May 15, 2006

(30) Foreign Application Priority Data
Apr. 8, 2003 (US) 60461083

(51) Int. Cl. A61K 33/24; A61K 31/29

(52) U.S. Cl. 424/653; 514/6; 514/503

(57) ABSTRACT

This invention relates to methods and compositions for the treatment of conditions associated with abnormal activity or secretion of the hormone gastrin. In particular the invention relates to the treatment of conditions associated with non-amidated gastrin. In one aspect there is provided a method of treatment or prophylaxis of a condition associated with elevated levels of non-amidated gastrin, comprising the step of administering to a mammal in need of such treatment an effective amount of a compound which has the ability to inhibit the binding of ferric ions to any one or more of glycine-extended gastrin₁₇ or progastrin or progastrin-derived peptides, but which does not inhibit the activity of amidated gastrin, thereby to inhibit the activity of non-amidated gastrins.

This invention relates to methods and compositions for the treatment of conditions associated with abnormal activity or secretion of the hormone gastrin. In particular the invention relates to the treatment of conditions associated with non-amidated gastrin.

### BACKGROUND OF THE INVENTION

This application claims priority from U.S. provisional patent application No. 60/461,083, the entire contents of which are incorporated herein by cross-reference.

All references, including any patents or patent applications, cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents forms part of the common general knowledge in the art, in Australia or in any other country.

Gastrin is a classical gut peptide hormone, which was identified originally as a stimulant of gastric acid secretion. It is produced principally by the G cells of the gastric antrum, and to a variable extent in the upper small intestine, with much lower amounts in the colon and pancreas. The related hormone cholecystokinin (CCK), which is responsible for pancreatic enzyme secretion, has the same C-terminal tetrapeptide amide as gastrin.

Like many other peptide hormones, the initial translation product of the gastrin gene is a large precursor molecule, preprogastrin (101 amino acids), which is converted to progastrin (80 amino acids, SEQ ID NO:1) by cleavage of the N-terminal signal peptide. Progastrin is processed further within secretory vesicles by endo- and carboxy-peptidases to yield glycine-extended gastrins. The C-terminus of glycine-extended gastrin₃₄ has the glycine extension removed and is then amidated by peptidyl α amidating mono-oxygenase, producing amidated gastrin₃₄ and further proteolytic cleavage results in mature amidated gastrin₁₇ (Gamide). Where the glycine extension is not cleaved there is no amidation of the peptide, and proteolytic cleavage results in the 18 amino acid glycine-extended form of gastrin₁₇ (Ggly). In healthy humans progastrin and the various forms of glycine-extended gastrins comprise less than 10% of circulating gastrins.

Progastrin and its glycine-extended derivatives have previously been regarded as physiologically inactive. However, data have been accumulating to suggest that gastrin precursors such as Glycine-extended gastrin stimulate proliferation in several cancer cell lines. The observation that CCK-2 receptor antagonists did not inhibit the growth response to Ggly suggests that novel receptors, distinct from classical gastrin receptors, are involved.

Amidated gastrins bind with high affinity to CCK-1 and CCK-2 receptors (previously referred to as CCK-A and CCK-B receptors, respectively), which can be differentiated by the substantially greater affinity of the CCK-1 receptor for CCK (Baldwin and Shulkes, 1998). Both receptors belong to the family of 7 transmembrane domain receptors, and share 50% identity in sequence. In addition both amidated and non-amidated gastrins bind to a low affinity receptor, often referred to as the CCK-C receptor, which is present in a variety of tissues, including neoplastic cell lines (Weinstock and Baldwin, 1988). The CCK-C receptor is unrelated in structure to the classical gastrin receptors, and belongs to the family of enzymes involved in β-oxidation of fatty acids (Baldwin, 1993).

The initial confusion over the type and number of CCK receptors expressed in the gastrointestinal tract has now been clarified. In the fundus of the stomach, CCK-2 receptors have been identified on parietal, enterochromaffin-like (ECL) and D cells, while in the antrum they are confined to D cells. CCK-1 receptors are present on fundic chief cells and antral D cells. In the human, pancreatic acini do not express significant numbers of functional CCK-1 receptors. The normal human pancreas does contain CCK-2 receptors, which are localised to the glucagon-producing cells of the pancreatic islets. The normal colon in general does not seem to express CCK-2 receptors.

Gamide is an important trophic factor for gastric epithelium, and is known to stimulate proliferation of the ECL cells of the stomach and proximal small intestine (Lehy, 1984), and gastric parietal cell migration (Kirton et al, 2002). This proliferative effect can result in carcinoid tumour formation secondary to prolonged hypergastrinaemia in conditions such as Zollinger-Ellison syndrome (Kidd et al, 1998). In contrast, Gamide does not appear to have a significant proliferative role in other regions of the gastrointestinal tract. However, Gamide does appear to act as a mitogen for the metaplastic ductular cells generated in vivo by ligation of rat pancreatic ducts (Rooman et al, 2001).

Growth effects of non-amidated gastrins have been demonstrated in colonic tissue, both in vivo and in vitro. For example, infusion of Ggly into gastrin-deficient mice increased the colonic proliferative index by 80%, but infusion of Gamide had no effect on the proliferative index. Transgenic mice over-expressing human progastrin in the liver have high concentrations of circulating progastrin, but normal Gamide concentrations. These mice have thickened colonic mucosa, with deeper crypts and an increased proliferative index in both proximal and distal colon compared to wild type mice (Wang et al, 1996). Similar results have been reported for transgenic mice over-expressing Ggly (Koh et al, 1999). Since these transgenic mice have had high progastrin exposure since infancy, it could be argued that the results reflect developmental and/or lifelong exposure.

Although recent studies with gastric carcinoma cell lines have demonstrated the presence of gastrin mRNA and processed and unprocessed gastrin, growth of the majority of these lines is not stimulated by exogenous Gamide (reviewed in Baldwin, 1995). Recently both Gamide and Ggly were shown to stimulate growth of three human gastric carcinoma cell lines. The effects of Gamide on AGS and IMGE cells were mediated by the CCK-2 receptor (Iwase et al, 1997; Hollande et al, 2001), while Gamide stimulated SIIA cells via a CCK-1-like receptor (Iwase et al, 1997). The observation that CCK-1 or CCK-2 receptor antagonists did not block the proliferative effect of Ggly suggested the involvement of a novel receptor (Iwase et al, 1997; Hollande et al, 2001).

Ggly stimulated growth of approximately 50% of the colorectal cell lines tested, via a receptor distinct from the CCK-2 receptor (Hollande et al, 1997; Stepan et al,1999; Litvak et al, 1999).

The presence of gastrins and their receptors in gastric adenocarcinomas remains a controversial issue. Gamide, progastrin, Ggly and the CCK-2 receptor have been detected in human gastric adenocarcinomas by immunohistochemistry, with an increased proportion of positive cells in the progression from intestinal metaplasia to adenocarcinoma (Henwood et al, 2001). However, CCK-2 receptor expression was detected in only 7% of gastric cancer samples (Okada et al, 1996; Reubi et al, 1999).

Early data suggested that some colorectal cancers and cell lines synthesised gastrin and expressed gastrin receptors, although the percentage of positive tumours varied greatly between groups. Moreover, the growth of some colorectal carcinoma cell lines was stimulated by exogenous Gamide, and could be inhibited by gastrin receptor antagonists (see Baldwin and Shulkes, 1998 for review). Reports of hypergastrinaemia in patients with colorectal cancer also raised the possibility that gastrin might act as an endocrine proliferative agent, with the source of gastrin remaining undefined.

More recent data support the view that Gamide and the CCK-2 receptor do not play a significant role in the growth of the majority of colorectal carcinomas. CCK-2 receptors were expressed in only a small subset (4%) of colorectal carcinomas, and the authors cautioned that the presence of receptors in non-malignant tissue contaminating the tumour could give rise to an over-estimate of receptor-positive tumours (Reubi et al, 1999). When taken together with the infrequent occurrence of both Gamide peptide production and CCK-2 receptor expression in human colorectal carcinomas, the animal data suggests that a role for Gamide may be limited to a small subset of tumours.

Recent studies on progastrin and Ggly confirm that it may be these peptides rather than Gamide that play a role in colorectal carcinogenesis. Colonic neoplastic tissue consistently synthesises progastrin, but is deficient in the processing of progastrin to Gamide. The degree of processing is quite variable between individual tumours. Activation of the gastrin gene in colonic mucosa cells may occur when carcinoma develops, as a result of mutations in the APC, β-catenin or k-ras genes.

The possible tumour-promoting effects of gastrin precursors have been studied in transgenic mice treated with carcinogens. After treatment with azoxymethane, progastrin-expressing transgenic mice had increased numbers and sizes of tumours compared to wild type mice. In addition, an increased proportion (42%) of tumours in the progastrin group were in the proximal colon compared to controls, in which the majority were in the distal colon (Singh et al, 2000b). These observations correlated well with findings of increased numbers of aberrant crypt foci in the progastrin group in an earlier study (Singh et al, 2000a). Even short-term exposure (4 weeks) of azoxymethane-treated rats to exogenous Ggly resulted in a significant increase in the number of aberrant crypt foci formed (Aly et al, 2001). The observation that exogenously administered Ggly (Aly et al, 2001) or endogenous transgenic production of progastrin (Koh et al, 1999) can potentially promote colorectal cancer by increasing the number of aberrant crypt foci suggests that early activation of the gastrin gene may similarly provide a tumour-enhancing environment via an autocrine pathway. A surprising recent observation that gastrin-deficient mice developed more colonic adenocarcinomas than wild-type mice after treatment with azoxymethane was interpreted in terms of an inhibitory role for Gamide in colorectal carcinogenesis (Cobb et al, 2002). This interpretation is consistent with the proposal that it is the non-amidated forms of gastrins that are responsible for the acceleration of colon carcinogenesis.

Gastrins may also modulate cell migration and invasiveness. The CCK-1 receptor antagonist loxiglumide reduced both the expression of matrix metalloproteinase-9 (MMP-9) and the invasiveness of 2 human pancreatic carcinoma cell lines (Hirata et al, 1996). Similar results have been obtained following Gamide treatment of a CCK-2 receptor-transfected subline of the human gastric cancer cell line AGS (Wroblewski et al, 2002). However, only non-amidated gastrins such as Ggly were able to stimulate migration of the human colorectal carcinoma cell line LoVo and the mouse gastric cell line IMGE-5 (Hollande et al, 2001).

Animal experiments provide strong evidence that non-amidated gastrins such as Ggly stimulate colonic mucosal growth, accelerate the early steps in colorectal carcinoma formation, and are elevated in the tumour and circulation of patients with colorectal cancer. The CCK-2 receptor appears to play a role in gastric and pancreatic neoplasia, and gastrin precursors may act as autocrine growth factors in colorectal carcinoma. Studies in gastrin knockout animals demonstrating a reduction in tumour incidence suggest that gastrin receptors may provide an additional target for prophylaxis or therapy for colorectal cancer.

Indeed, immunization against gastrin has been suggested as a method of treatment or prevention of gastrin-dependent pancreatic tumours, or hypergastrinaemic conditions; see for example PCT/US02/00685, PCT/US99/10751, PCT/US98/09957 and PCT/US97/02029 by Aphton Corporation. Other therapeutic approaches include the use of compounds which target the gastrin-releasing peptide receptor or the gastrin and/or CCK receptor, or which inhibit expression of gastrin. However, it may be preferable for therapies aimed. at the gastrins to be targeted to the relevant gastrin/gastrin receptor combination.

As far as we are aware, there have not been any attempts to direct such therapies to specific inactivation of Ggly, or of blockade of the interaction between Ggly and its receptor. Although PCT/US97/02029 discloses the use of antibodies to inhibit the activity of Ggly, the immunogens used elicited antibodies against both Gamide and Ggly. These immunogens are disclosed in U.S. Pat. No. 5,023,077, and were designed to avoid induction of antibodies specific for or cross-reactive with gastrin₃₄, which might induce undesirable side effects by blocking physiological functions of gastrin₃₄. The antibodies resulting from immunization with such immunogens targeted an epitope in gastrin17 (Gamide) which is antigenically and immunogenically distinct from the structure of gastrin₃₄. This epitope consists of the amino acid sequence from residues 1 to 12 inclusive of Gamide.

Bismuth salts have been used for over two centuries for the treatment of various gastrointestinal disorders (Gorbach, 1990) particularly gastric and duodenal ulcer. Bismuth salts have antibacterial and antiproliferative effects (Van Orjen et al, 2000; Marshall et al, 1987), but the mechanism of action of these salts in the treatment of gastrointestinal diseases is still unknown.

Bismuth salts have also been proposed to be useful in the treatment of corneal and dermal wounds and of halitosis, by virtue of their antimicrobial activity against anaerobes such as Campylobacter rectus and Treponema denticola (U.S. Pat. No. 4,626,085). Iodiscorbate compounds of formula XISrC₆8₅O₆, in which X is bismuth, potassium or zinc, have been proposed to be useful in the treatment of cancer by dissociating an ozonide in the TATA box of a DNA oncogene (U.S. Pat. No. 6,294,678). A preferred compound is BiRSrC₆8₅O₆. The compounds disclosed in this patent are very different from the simple bismuth salts that are conventionally used.

Because Ggly is implicated in a number of pathological conditions, there is a need in the art for agents which are able to specifically inhibit the activity of this hormone. However, the receptor for non-amidated gastrin has not yet been isolated and identified, and no high-affinity antagonists of this hormone are available.

### SUMMARY OF THE INVENTION

Ggly is able to bind ferric ions (Baldwin, Curtain et al, 2001). We have now found that ferric ion binding modulates the biological activity of Ggly. Moreover, the amino acid residue corresponding to Glu7 of Ggly is essential for, and one or more of Glu8, Glu9 and Glu10 contribute to, the ability of Ggly to bind ferric ions. We therefore propose that the natural ligand for the putative high-affinity receptor(s) for Ggly may be the complex between Ggly and ferric ions, rather than Ggly itself as we previously proposed.

We have also previously found that non-amidated gastrins could stimulate cell proliferation independently of the receptors for amidated gastrins. We have now surprisingly found that certain trivalent metal ions have the ability to specifically block biological activity of Ggly. We have also now demonstrated that non-amidated amino acid sequences from progastrin and Ggly as short as the heptapeptides EEEEEAY (SEQ ID NO:2)and LEEEEEA (SEQ ID NO:3) are biologically active, and that this activity is modulated by interaction of the peptide with ferric ions.

In a first aspect, there is provided a method of treatment and/or prophylaxis of a condition associated with elevated levels of non-amidated gastrin, comprising the step of administering to a mammal in need of such treatment an effective amount of a compound which has the ability to inhibit the binding of ferric ions to the glycine-extended gastrin₁₇ molecule and/or to progastrin, but which does not inhibit the activity of amidated gastrin, and thereby to inhibit the activity of non-amidated gastrins.

Preferably the compound reduces or inhibits the binding of ferric ions to glutamate 7 of the glycine-extended gastrin₁₇ molecule. In one embodiment, the binding of ferric ions to glutamate 8 and glutamate 9 of the glycine-extended gastrin₁₇ molecule is also inhibited.

In one embodiment the compound is a metal ion, or a pharmaceutically-acceptable salt or complex thereof, which is able to occupy the ferric ion binding site of non-amidated gastrins, and thereby to block their biological activity.

The metal may be any metal ion capable of occupying the ferric ion binding site of a non-amidated gastrin, with the provisos that
(i) when the condition is one caused by Helicobacter pylori infection, the metal ion is not bismuth, and
(ii) when the condition is cancer, the salt or complex is not BiISrC₆H₅O₆.

Preferably the metal ion is Bi³⁺ or Ga³⁺.

In another embodiment the compound is an exchange-inert complex between a non-amidated gastrin and either Co (III) or Cr (III) ions. Methods for preparation of exchange-inert complexes are known in the art.

In a third embodiment the compound is a pharmaceutically-acceptable chelating agent with a high degree of specificity for ferric ions. Many such agents are known, and the person skilled in the art will readily be able to assess whether a given chelating agent is suitable. Membrane-impermeable chelating agents are preferred, because it is desirable to block the effect of extracellular non-amidated gastrin, such as Ggly, without interfering with intracellular ferric ion-dependent processes. It is contemplated that the membrane-impermeable chelators such as ethylene diamine tetracetic acid (EDTA) and diethylene triamine pentacetic acid (DTPA) will be useful for the purposes of the invention. However, it must be emphasised that membrane-permeable chelators such as clioquinol may also be useful, depending on the target tissue. For example, in the case of colorectal carcinoma interference with ferric ion-dependent processes within the cancer cells would not present a problem, since the ultimate objective would be to kill the cancer cells.

Preferably the compound does not have a significant inhibitory effect on Gamide-induced inositol phosphate production in cells which express the CCK-2 receptor, and/or on cellular proliferation in cells which express the CCK-2 receptor.

Suitable pharmaceutically-acceptable bismuth salts include colloidal bismuth subcitrate (CBS), bismuth subcitrate, bismuth citrate, bismuth salicylate, bismuth subsalicylate, bismuth subnitrate, bismuth subcarbonate, bismuth tartrate, bismuth subgallate, tripotassium dicitrato bismuthate and bismuth aluminate. Preferably the salt is colloidal bismuth subcitrate (CBS), tripotassium dicitrato bismuthate, bismuth subcitrate, or bismuth subsalicylate. More preferably the salt is CBS or tripotassium dicitrato bismuthate. Most preferably the salt is CBS, which has been extensively investigated and demonstrated to have low toxicity: see references cited in U.S. Pat. No. 6,426,085. Other bismuth-containing compounds which may be used in the present invention are those described in U.S. Pat. Nos. 4,801,608 and No. 4,153,685, both of which are expressly incorporated herein by reference. It will be appreciated that a combination of two or more bismuth salts or other bismuth-containing compounds may be used.

The condition associated with elevated levels of non-amidated gastrin may be any pathological condition which exhibits any one or more of the following which contribute to one or more symptoms of the condition: increased levels of non-amidated gastrin in the blood, an increase in the proportion of non-amidated gastrin in the blood compared with amidated gastrin, the presence of tumour cells which secrete non-amidated gastrin, and an increased rate of secretionor level of activity of non-amidated gastrin, such as Ggly.

The condition may involve cell proliferation, cell migration, or acid secretion by cells which are responsive to non-amidated gastrin.

Preferably the condition is selected from the group consisting of gastrin-producing tumours, such as colorectal carcinomas, gastrinomas, islet cell carcinomas, lung cancer, ovarian cancer, pituitary cancer and pancreatic cancer, or from other conditions in which serum gastrins are elevated, such as atrophic gastritis; G cell hyperplasia; pernicious anaemia; and renal failure; or from other conditions affecting the gastrointestinal mucosa, such as ulcerative colitis.

Since non-amidated gastrins are known to act as growth factors in the colonic mucosa, specific inhibitors of these gastrins are useful for the treatment of disorders of gastrointestinal proliferation, such as ulcerative colitis and gastrointestinal cancers. In particular it is known that any prolonged elevation of gastrin levels increases the risk of colon cancer or pancreatic cancer. Thus the invention is applicable to the treatment or prevention of these conditions, especially in individuals at elevated risk thereof. The risk of colon cancer is also elevated in individuals on diets high in fat or meat and in individuals with adenomatous polyposis coli, with familial adenomatous polyposis, or with a family history of colon cancer, who are therefore also suitable candidates for prophylactic treatment according to the invention. It has also been reported that loss of imprinting of IGF-2 is associated with colon cancer, and may provide a basis for a blood test to identify those at an increased risk of this cancer (Cui et al, 2003).

Non-amidated gastrins also potentiate the stimulation of acid secretion by amidated gastrins, so specific inhibitors of non-amidated gastrins are also useful for the treatment of excessive acid production in patients with conditions such as gastrointestinal ulcers, gastro-oesophageal reflux, gastric carcinoid, or Zollinger-Ellison syndrome, including those being treated with proton pump inhibitors or H₂ blockers; however, for this purpose it is to be clearly understood that the metal ion is not bismuth.

Since non-amidated gastrins are known to act as growth factors in the intestinal mucosa, our findings also indicate that GGly or its active fragments could themselves be used in situations where the gastrointestinal tract would benefit from additional proliferative stimuli, for example following massive small bowel resection or during total parenteral nutrition.

Accordingly, in one aspect there is provided a peptide which is a fragment of a non-amidated gastrin and which
(a) comprises at least glutamate residue 7 of the -(Glu)₅- sequence of non-amidated gastrin, and
(b) which is capable of binding one or more ferric ions, with the proviso that the peptide is not full length Ggly, full length glycine-extended gastrin or full length progastrin.

In one embodiment, the amino acid sequence consists of amino acids 5 to 14 of the amino acid sequence of Ggly.

In one embodiment there is also provided a complex comprising (a) a non-amidated gastrin, or a peptide fragment thereof and (b) a trivalent metal ion, such as Bi³⁺ or Ga³⁺.

In a further embodiment there is also provided a method of promoting intestinal function, comprising the step of administering a peptide fragment and/or a complex to a subject in need of such treatment.

In another embodiment there is provided a method of screening of candidate metal ion-binding compounds for ability to modulate the activity of non-amidated gastrins, comprising the steps of assessing the ability of the compound to inhibit binding of ferric ions to a non-amidated gastrin and/or assessing the ability of the compound to modulate proliferation and/or migration of cells of a gastric mucosal cell line in response to a non-amidated gastrin.

Also provided is the use of a compound which has the ability to inhibit the binding of ferric ions to glycine-extended gastrin₁₇ or to progastrin, but which does not inhibit the activity of amidated gastrin, in the manufacture of a medicament for the treatment or prophylaxis of a condition associated with elevated levels of non-amidated gastrin.

In another embodiment there is provided a use of a peptide fragment described above or a complex in the manufacture of a medicament for promoting intestinal function.

The invention represents a novel and unexpected method of blocking the biological actions of non-amidated gastrins. Occupation of the metal ion-binding site of non-amidated gastrins by bismuth or other metal ions of the invention prevents the binding of ferric ions, and so renders the peptide inactive. The major advantage of this approach is the specificity of inactivation, since bismuth binding has no effect on the binding of amidated gastrins to the CCK-2 receptor, or on their bioactivity. At the low concentrations of bismuth ions required for binding to non-amidated gastrins there is expected to be little interference with other biological processes.

The mammal may be a human, or may be a domestic or companion animal. While it is particularly contemplated that the compounds of the invention are suitable for use in medical treatment of humans, they are also applicable to veterinary treatment, including treatment of companion animals such as dogs and cats, and domestic animals such as horses, cattle and sheep, or zoo animals such as felids, canids, bovids, and ungulates.

Methods and pharmaceutical carriers for preparation of pharmaceutical compositions are well known in the art, as set out in textbooks such as Remington's Pharmaceutical Sciences, 20th Edition (2000), Williams & Williams, USA.

The compounds and compositions described may be administered by any suitable route, and the person skilled in the art will readily be able to determine the most suitable route and dose for the condition to be treated. Dosage will be at the discretion of the attendant physician or veterinarian, and will depend on the nature and state of the condition to be treated, the age and general state of health of the subject to be treated, the route of administration, and any previous treatment which may have been administered.

The carrier or diluent, and other excipients, will depend on the route of administration, and again the person skilled in the art will readily be able to determine the most suitable formulation for each particular case.

Animal models of relevant cancers are known, and may be used in the evaluation of efficacy of compounds of the invention (see for example Seimann, 1987; Aly et al, 2001).

### BRIEF DESCRIPTION OF THE FIGURES

FIG. 1 shows the structures of glycine-extended gastrin₁₇ and related peptides:
Ggly (SEQ ID NO:4)
Gamide (SEQ ID NO:5)
Ggly1-4 (SEQ ID NO:6)
Ggly1-11 SEQ ID NO:7)
Ggly12-18 SEQ ID NO:8)
Ggly5-18 SEQ ID NO:9)
GglyE6A SEQ ID NO:10)
GglyE7A SEQ ID NO:11)
GglyE8-10A SEQ ID NO:12)
GglyE6-10A SEQ ID NO:13).
The structures of N- and C-terminally truncated peptides and of alanine-substituted peptides derived from glycine-extended gastrin₁₇ (Ggly) are shown. Amino acids are shown in the one letter code, and Z represents a pyroglutamate residue. The -(Glu)₅- sequence is highlighted in bold. In Ggly mutants alanine residues, which replace some or all of the original glutamate residues, are underlined. Glycine-extended gastrin₁₇ (SEQ ID NO: 4) corresponds to residues 55-72 of mature progastrin₁₋₈₀ (SEQ ID NO:1).

FIG. 2 shows the parameters of the final 20 NMR structures of Ggly. All parameters are plotted as a function of residue number. A. Upper bound distance restraints used in the final round of structure refinement. Long-range (|i-j|>5), medium-range (2≦|i-j|≦5), and sequential NOE are shown in dark grey, grey and black respectively. NOEs are counted twice, once for each proton involved. B. Root Mean Standard Deviations (RMSD) from the mean structure for the backbone heavy atoms (nitrogen, α-carbon and carbon). C and D. Angular order parameters (S) for the backbone (φ and ψ, respectively) dihedral angles were calculated as described previously (30, 31).

FIG. 3 shows a stereo view of the backbone heavy atoms of Ggly. The final 20 structures of Ggly in aqueous solution were determined from two-dimensional NMR data, and the backbone heavy atoms (N, C^{α} and C) superimposed over the well-defined (S₁₀₁ and S_{Ψ}>0.9) region of the molecule.

FIG. 4 shows the orientation of hydrophobic residues of Ggly. The side chains of the -(Glu)₅- sequence (E6 to E10) are shown in dark grey. The backbone is shown in light grey.

FIG. 5 shows the effect of ferric ions on the ¹H NMR spectrum of Ggly, as illustrated by a contour plot (upper panel) and a stack plot of the Glu projections (lower panel) of the fingerprint region of the two-dimensional ¹H total correlation spectroscopy spectrum of Ggly (2.5 mM in 10% DMSO/10% ²H₂0/80% H₂0, pH 5.3). The resonances of the 5 glutamate residues at positions 6-10 in the Ggly sequence are indicated before (A), and after the addition of 1 (B) or 2 (C) equivalents of ferric ions.

FIG. 6 shows that glutamate residue 7 of Ggly acts as a metal ion ligand. The quenching by ferric ions of the tryptophan fluorescence of Ggly-derived peptides with single (A) or multiple (B) alanine substitutions was measured. The peptides were as follows: Ggly (O), GglyE6A (▾), GglyE7A (▴), GglyE6-10A (●), GglyE8-10A (▪). The buffer was 10 mM sodium acetate, pH 4.0, containing 100 mM NaCl and 0.005% Tween 20. Values of the stoichiometry and the apparent dissociation constant were obtained from the intercept and slope, respectively, of linear transformations of the data for peptides with single (C) and multiple (D) alanine substitutions by least squares fitting with the program Sigmastat. The results of at least 4 independent experiments were combined to obtain the mean values presented in Table 2. In all cases substitution of the glutamate at position 7 of Ggly resulted in a reduction of one in the number of bound ferric ions.

FIG. 7 shows that glutamate residues 6-10 are important for Ggly-induced biological activity. The effect of fragments of the Ggly sequence (1 nM) on proliferation (A) or migration (B) of IMGE-5 cells was measured by MTT and wound healing assays, respectively. Data are means±S.E. from at least three independent experiments. Statistical significance relative to the control (*, p<0.05, **, p<0.01) was assessed by one-way analysis of variance, followed by Bonferroni's t-test.

FIG. 8 shows that glutamate residue 7 is essential for Ggly-induced biological activity. The effect of amino acid substitutions of the Ggly sequence (1 nM) on proliferation (A) or migration (B) of IMGE-5 cells was measured by MTT and wound healing assays, respectively. Data are means±S.E. from at least three independent experiments. Statistical significance relative to the control (*, p<0.05, **, p<0.01) was assessed by one-way analysis of variance, followed by Bonferroni's t-test.

FIG. 9 shows that ferric ions are essential for Ggly-induced biological activity. The effect of the iron chelator desferrioxamine (DFO, 1 μM) on IMGE-5 cell proliferation induced by Ggly or Gamide (A) or migration induced by Ggly (B) was measured by MTT and wound healing assays, respectively. Data are means±S.E. from at least three independent experiments. Statistical significance relative to the control (*, p<0.05, **, p<0.01), or to the appropriate value without DFO (*, p<0.01), was assessed by one-way analysis of variance, followed by Bonferroni's t-test.

FIG. 10 shows that iron chelators other than DFO also inhibit Ggly-induced biological activity. The effect of the iron chelators clioquinol (A) or doxorubicin (B) on IMGE-5 cell proliferation induced by Ggly was measured by [³H]-thymidine incorporation. Data are means ±S.E. from at least three independent experiments. Statistical significance relative to the control (*, p<0.05, **, p<0.01), or to the appropriate value without chelator (##, p<0.01) , was assessed by one-way analysis of variance, followed by Bonferroni's t-test.

FIG. 11 illustrates the binding of bismuth ions by gastrins. The tryptophan fluorescence (A) of 10.35 μM Gamide, in 10 mM sodium acetate (pH 4.0) containing 100 mM NaCl and 0.005% Tween 20, was quenched in the presence of increasing concentrations of trivalent bismuth (Δ) or ferric (●) ions, but not chromium (▾) ions. (B) The values for the fraction of occupied sites (Fa) for the binding of bismuth ions to Gamide (▴) or to Ggly (Δ) were calculated, and fitted to various binding models, as described in Materials and Methods. The lines of best fit for Gamide (-, Kd=8.6±1.2 μM) or Ggly ( - - - , Kd=7.3±1.0 μM) were obtained with a model with two independent binding sites with identical affinities. These values were combined with the values from four other experiments to obtain the mean values presented in Table 3.

FIG. 12 shows the effect of bismuth ions on the ¹H NMR spectrum of Ggly. Contour plot (upper panel) and stack plot of the Glu projections (lower panel) of the fingerprint region of the two-dimensional ¹H total correlation spectroscopy spectrum of Ggly (2.5 mM in 10% DMSO/10% ²H₂O/80% H₂O, pH 5.3) are shown. The resonances of the five glutamate residues at positions 6-10 in the Ggly sequence are indicated before (A), and after the addition of 4 (B) or 25 (C) moles/mole of bismuth ions.

FIG. 13 shows that bismuth ions selectively inhibit Ggly-induced inositol phosphate production. The effect of bismuth ions at a concentration of 2, 8 or 32 moles/mole on 10 nM Ggly-induced (A) or Gamide-induced (B) inositol phosphate production was measured in HT29 cells (A) or in COS cells transiently transfected with the CCK-2 receptor (B). Data are means±S.E. of triplicates from one experiment, and similar results were obtained in four independent experiments. Statistical significance relative to the control (*, p<0.05, **, p<0.01) or to the Ggly-stimulated sample (##, p<0.01) was assessed by one-way repeated measures analysis of variance, followed by Bonferroni's t-test.

FIG. 14 shows that bismuth ions inhibit Ggly-induced cell proliferation. The effect of bismuth ions at a concentration of 2, 8 or 32 moles/mole on 10 nM Ggly-induced proliferation of HT29 cells was measured in MTT assays. The figure shows means±S.E. of triplicates from one experiment, and similar results were obtained in four independent experiments. Statistical significance relative to the control (*, p<0.05, **, p<0.01) or to the Ggly-stimulated sample (##, p<0.01) was assessed by one-way repeated measures analysis of variance, followed by Bonferroni's t-test.

FIG. 15 shows that bismuth ions inhibit Ggly-induced cell migration. The effect of bismuth ions at a concentration of 2, 8 or 32 moles/mole Ggly on 10 nM Ggly-induced migration of IMGE-5 cells was measured in wound healing assays over 17 (open bars) or 24 hours (closed bars). The figure shows means±S.E. from four independent experiments. Statistical significance relative to the Ggly-treated sample (*, p<0.05, **, p<0.01) was assessed by one-way repeated measures analysis of variance, followed by Bonferroni's t-test.

FIG. 16 shows the effect of bismuth ions on the binding of gastrins to their receptors. The effect of bismuth ions at a concentration of 2, 8 or 32 moles/mole on specific binding of [¹²⁵I-Ggly to the Ggly receptor on IMGE-5 cells (A) or of [¹²⁵I]-BH-CCK₈ to CCK-B receptors on transiently transfected COS-7 cells was measured. The figure shows means±S.E. of triplicates from one experiment, and similar results were obtained in three independent experiments. Statistical significance relative to the control (*, p<0.05, **, p<0.01) was assessed by one-way repeated measures analysis of variance, followed by Bonferroni's t-test.

FIG. 17 shows a comparison of the ability of several divalent (grey bars) and trivalent (black bars) metal ions to compete with ⁵⁹Fe³⁺ for binding to the polyglutamate binding site common to both progastrin₆₋₈₀ and Ggly.

FIG. 18 shows that ferric ions are essential for Ggly-induced biological activity in vivo. The effect of the iron chelator desferrioxamine (DFO) on Ggly-induced proliferation in the rectal mucosa of rectally defunctioned rats was measured by counting metaphase-arrested nuclei as described previously (Aly et al., 2001). Data are means±S.E. from at least 7 animals per group. Statistical significance relative to the control (*, p<0.05, ***, p<0.001), or to the appropriate value without DFO (##, p<0.01), was assessed by one-way analysis of variance, followed by Bonferroni's t-test.

FIG. 19 shows that bismuth ions can block Ggly-induced biological activity in vivo. The effect of bismuth ions, administered by oral gavage as colloidal bismuth subcitrate, on Ggly-induced proliferation in the rectal mucosa of rectally defunctioned rats was measured by counting metaphase-arrested nuclei as described previously (Aly et al., 2001). Data are means±S.E. from at least 7 animals per group. Statistical significance relative to the untreated control animals (***, p<0.001), or to the Ggly-stimulated animals (#*#, p<0.001), was assessed by one-way analysis of variance, followed by Bonferroni's t-test.

FIG. 20 shows that glutamate residue 7 of Gamide acts as a metal ion ligand. The quenching by ferric ions of the tryptophan fluorescence (A) of Gamide-derived peptides with single or double alanine substitutions was measured. The peptides were as follows: Gamide (◯), GamideE7A (●) (SEQ ID NO:14), GamideE8, 9A (▪) (SEQ ID NO:15). The buffer was 10 mM sodium acetate, pH 4.0, containing 100 mM NaCl and 0.005% Tween 20.

FIG. 21 shows that Glu7-9 are not essential for binding of Gamide to the CCK-2 receptor on transfected COS-7 cells.

FIG. 22 shows that Glu7-9 are not essential for binding of Gamide to the CCK-2 receptor on Jurkat cells. The ability of increasing concentrations of Gamide (A), GamideE7A (B), and Gamide E8,9A (C) to compete with [¹²⁵I]-BH-CCK₈ (150 μM, 100,000 cpm) for binding to the human CCK-2 receptor on Jurkat cells was measured. Points are the means±S.E. of triplicates, expressed as a percentage of the value obtained in the absence of peptide competitor.

FIG. 23 shows that Glu7-9 are not essential for Gamide-induced inositol phosphate production. Points are the means±S.E. of triplicates, expressed as a percentage of the value obtained in the absence of stimulation. Statistical significance relative to the control (**, p<0.001), was assessed by one-way analysis of variance, followed by Bonferroni's t-test.

FIG. 24 shows that ferric ions are not essential for binding of Gamide to the CCK-2 receptor or for Gamide-induced inositol phosphate production. The ability of increasing concentrations of Gamide to compete with [¹²⁵I]-BH-CCK₈ (150 pM, 100,000 cpm) for binding to the human CCK-2 receptor on (A) transiently transfected COS-7 cells or (B) Jurkat cells in the presence (◯) and absence (●) of 1 μM DFO was measured as described for FIG. 21 legend. The values for IC₅₀ and for the ordinate intercept used to construct the indicated lines of best fit were as follows: (A) Gamide (-), 56.6 nM, 81.1%; Gamide+DFO ( - - - ), 57.8 nM, 81.7%. (B) Gamide (-), 2.6 nM, 99.6%; Gamide+DFO ( - - - ), 2.6 nM, 94.3%. The values shown in FIG. 24 were combined with the data from at least two other experiments to obtain the mean values presented in Table 9. (C) The effect of 10 μM AlF₄⁻ (AlF), or 10 nM Gamide in the presence and absence of 1 μM DFO, on inositol phosphate production in COS-7 cells transiently transfected with the human CCK-2 receptor was measured as described for FIG. 23.

FIG. 25 shows that the nonapeptide LE₅AYG (SEQ ID NO:16) is active, and that ferric ions are essential for activity. The effect of Ggly (SEQ ID NO:4)(white bars) or the nonapeptide LE₅AYG (amino acids 5-13 of Ggly, grey bars, 100 nM) on proliferation (A) or migration (B) of IMGE cells was measured in thymidine uptake (A) or wound healing (B) assays, in the absence or presence of the iron chelator desferrioxamine (DFO, hatched bars). Wound size was measured at time zero and after 24 h treatment. Data are means±S.E. of two independent experiments, each in triplicate. Statistical significance relative to the control (*, p<0.05, **, p<0.01) or to the peptide-stimulated sample (#, p<0.05, ##, p<0.01) was assessed by one-way analysis of variance, followed by Bonferroni's t-test.

FIG. 26 shows that the potency of Ggly fragments depends on chain length. The effects of increasing concentrations of Ggly (SEQ ID NO:4) (●) or of the indicated fragments of the nonapeptide LE₅AYG (SEQ ID NO:16) (A) or the heptapeptide E₅AY (SE ID NO:2) (B) on proliferation of IMGE cells were measured in thymidine uptake assays. Points represent the means of five independent experiments, each in duplicate. Lines of best fit to the equation
s=100+S·C/(ED₅₀+C)
were generated with the program Sigmaplot. S.E. bars are only shown on the maximum and minimum data sets for clarity. The best fit values for ED₅₀ and the maximum % stimulation S used to construct the lines are presented in Table 1.

FIG. 27 shows that the activity of Ggly fragments of seven residues or more was inhibited by DFO, but that the activity of shorter fragments was not dependent on ferric ions. The effect of Ggly fragments (100 nM) on proliferation of IMGE cells was measured in thymidine uptake assays, in the absence (black bars) or presence (grey bars) of the iron chelator desferrioxamine (DFO).
LE₅AYG (SEQ ID NO:16)
LE₅A (SEQ ID NO:3)
E₅AY (SEQ ID NO:2)
LE₅ (SEQ ID NO:17)
E₅A (SEQ ID NO:18)
E₅ (SEQ ID NO:19)
Data are means±S.E. of at least three independent experiments, each in duplicate. Statistical significance relative to the unstimulated control (*, p<0.05, **, p<0.01), was assessed by one-way analysis of variance, followed by Bonferroni's t-test. Statistical significance of each peptide with DFO relative to the corresponding treatment without DFO (##, p<0.01) was determined by t-test.

FIG. 28 shows that Glu9 and GlulO (numbering as for Ggly) act as ferric ion ligands. The amide region of the one-dimensional 1H NMR spectrum of LE₅AYG (A) at 500 MHz, or E₅AY (B) or E₅ (C) at 600 MHz, is shown. Peptide concentrations were approximately 2 mM in 95% H₂O/5% ²H₂O (A) or 90% H₂O/10% ²H₂O (B,C), pH 5.3. The N-terminal amino group is not visible because of fast exchange; the remaining resonances are fortuitously in the same order as the peptide sequences. Addition of 1 or 2 moles/mole of ferric citrate to the nonapeptide LE₅AYG caused a selective downfield shift in the resonances due to Glu9 and Glu10 (arrowed, numbering as for Ggly). Addition of ferric citrate to the pentapeptide E₅ did not shift the glutamate resonances.

FIG. 29 shows that Ggly fragments also bind two ferric ions. The absorption spectrum of (A) the nonapeptide LE₅AYG or (B) the heptapeptide E₅AY (100 μM in 10 mM sodium acetate (pH 4.0) containing 100 mM NaCl and 0.005% Tween 20) in the absence (solid line) and presence of ferric ions at a ratio of 1 (dashed line) or 2 (dotted line) mole per mole was measured at 25° C. The spectrum was corrected for the change observed when the same concentration of ferric ions was added to 5% DMSO in buffer alone. Addition of aliquots of ferric chloride to (C) the nonapeptide or (D) the heptapeptide resulted in a linear increase in the absorption minimum at 247 nm up to a molar ratio of approximately 2 (●). A similar stoichiometry was observed from titrations at the absorption maximum of 275 nm (▪). The mean values for stoichiometry and maximum increase for the three tyrosine-containing peptides LE₅AYG, LE₅AY and E₅AY from three independent experiments are presented in Table 11.

FIG. 30 shows that Ggly fragments bind ferric ions with mM affinity. The reciprocal of the change in chemical shift (Δδ) of the GlulO NH resonance of the nonapeptide LE₅AYG (●) or the octapeptide LE₅AY (▪) was plotted against the reciprocal of the concentration of added ferric citrate ([Fe³⁺]) as described in the Materials and Methods section. Lines of best fit were obtained by linear regression with the program Sigmaplot, and values of the apparent dissociation constant K_{d}(LE₅AYG, 7.0 mM, r²=0.995; LE₅AY, 5.4 mM, r²=0.996) were calculated by dividing the slope by the ordinate intercept.

### DETAILED DESCRIPTION OF THE INVENTION

Amidated and non-amidated gastrins elicit different biological effects via distinct receptors in different tissues. Amidated gastrin₁₇ (Gamide) stimulates gastric acid secretion and the development of gastric carcinoids, whereas glycine-extended gastrin₁₇ (GGly) stimulates proliferation of the colonic mucosa and the development of colorectal cancers. Glycine-extended gastrin₁₇ binds two ferric ions with high affinity (Baldwin et al, 2001). We have now investigated the identity of the iron ligands and the role of ferric ions in biological activity, and we have determined the solution structure of glycine-extended gastrin₁₇ by NMR spectroscopy. The metal ion ligands were then identified by observing the fluorescence and NMR spectral changes following the addition of one or two equivalents of ferric ions to solutions of Ggly or Ggly mutants with substitutions of one or more of the amino acids involved in ferric ion binding. The respective abilities of Ggly and different Ggly fragments or Ggly mutants to stimulate proliferation and migration of the gastric epithelial cell line IMGE-5 were then compared. We found that Glu7 is critical for binding the first ferric ion, and that Glu8 and Glu9 are involved in binding the second ferric ion. Moreover the complete lack of activity in both assays of a Ggly mutant in which Glu7 was replaced by Ala (GglyE7A), and the inhibition of Ggly activity by the iron chelator desferrioxamine (DFO), indicate that ferric ion binding is essential for biological activity.

The prohormone progastrin is produced by G cells located within the gastric antrum, and is processed to shorter peptides, such as glycine-extended gastrin (Ggly) and amidated gastrin (Gamide), whose sequences are shown in FIG. 1. Until recently, it was thought that amidation of the carboxy terminus of gastrin was essential for biological activity, and that the C-terminal tetrapeptide was the minimum biologically active fragment (Dockray et al, 1999). However, we and others have reported that Ggly and progastrin are able to induce proliferation and migration of various cell lines in vitro (Seva et al, 1994; Hollande et al, 1997; Stepan et al, 1999; Hollande et al, 2001; Kermorgant et al, 2001), and proliferation of the colonic mucosa in vivo (Koh et al, 1999). In addition Ggly acts synergistically with Gamide in the stimulation and maintenance of elevated gastric acid production (Higashide et al, 1996).

We recently reported that Ggly specifically bound two trivalent ferric ions (Baldwin, Curtain et al, 2001), and we show herein that binding of ferric ions is essential for biological activity of GGly. Because bismuth ions are also trivalent, we hypothesised that gastrins might also bind bismuth ions, and that this might influence biological activity. In order to investigate this question, we have studied the nuclear magnetic resonance (NMR) and fluorescence spectra of gastrins in the presence of bismuth salts. We have also investigated the effect of bismuth ions on the binding of gastrins to their receptors, and on gastrin-induced inositol phosphate production, cell proliferation and cell migration. Our results indicate that bismuth ions selectively inhibit the biological activity of Ggly both in vitro and in vivo, but have little effect on the actions of Gamide. This novel effect of bismuth has implications for treatment of peptic ulcer disease, colitis and colorectal cancer.

It is to be clearly understood that this invention is not limited to the particular materials and methods described herein, as these may vary. It is also to be understood that the terminology used herein is for the purpose of describing particular embodiments only, and it is not intended to limit the scope of the present invention, which will be limited only by the appended claims.

Definitions

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

As used herein, the singular forms “a”, “an”, and “the” include the corresponding plural reference unless the context clearly dictates otherwise. Thus, for example, a reference to “an enzyme” includes a plurality of such enzymes, and a reference to “an amino acid” is a reference to one or more amino acids.

Where a range of values is expressed, it will be clearly understood that this range encompasses the upper and lower limits of the range, and all values in between these limits.

It will be clearly understood that for the purposes of this specification the term “non-amidated gastrin” is to be understood to be synonymous with any form of progastrin-derived peptide which contains the five consecutive glutamates of progastrin, but does not contain a phenylalanine-amide at its C-terminus. The term thus encompasses progastrin itself, “glycine-extended gastrin₁₇”, herein referred to as “Ggly”, both N- and C-terminally extended forms of glycine-extended gastrin₁₇, and also shorter peptides derived from the progastrin sequence between residues 55 and 72. Amidated gastrin₁₇ is referred to herein as “Gamide”.

The expression “elevated non-amidated gastrin” is to be understood to mean that the blood levels, rate of secretion or activity of Ggly are significantly higher than those in a normal subject of comparable age, sex and weight.

The expression “high degree of specificity for ferric ions” refers to the ability of a chelator to bind to ferric ions with affinity and/or ion selectivity comparable to the chelators exemplified in the specification.

It will be understood that the term “fragment” as used herein in relation to a peptide is intended to encompass a region of contiguous sequence from a full-length peptide and to exclude the full-length peptide, but is not intended to imply that the fragment is necessarily produced by cleavage of a full-length peptide. It will be understood that a “fragment” may also include a peptide which consists essentially of a region of contiguous sequence of a full-length peptide, as described above, but in which there are one or more amino acid substitutions, additions or deletions, provided that the substitutions, additions or deletions do not affect the ability of the fragment to bind one or more ferric ions. Given the availablity of modern methods such as solid-phase synthesis or site-directed mutagenesis, it is well within the capacity of the person skilled in the art to make and test a large number of such variants without undue effort.

In determining whether a particular peptide is “capable of binding ferric ions”, a person of skill in the art may utilise fluorescence spectroscopy as described in the specification. Generally, the terms “treating”, “treatment” and the like are used herein to mean affecting a subject, tissue or cell to obtain a desired pharmacological and/or physiological effect. The effect may be prophylactic in terms of completely or partially preventing a disease or sign or symptom thereof, and/or may be therapeutic in terms of a partial or complete cure of a disease. “Treating” as used herein covers any treatment of, or prevention of disease in a vertebrate, a mammal, particularly a human, and includes preventing the disease from occurring in a subject who may be predisposed to the disease, but has not yet been diagnosed as having it; inhibiting the disease, ie., arresting its development; or relieving or ameliorating the effects of the disease, ie., cause regression of the effects of the disease.

Unless defined otherwise, all technical and scientific terms used herein have the same meaning as commonly understood by one of ordinary skill in the art to which this invention belongs. Although any materials and methods similar or equivalent to those described herein can be used to practice or test the present invention, the preferred materials and methods are now described.

Abbreviations used herein are as follows:
CCK cholecystokinin
CRC colorectal carcinoma
DFO desferrioxamine
DMEM Dulbecco's modified Eagle's medium
FBS fetal bovine serum
Gamide amidated gastrin₁₇
Ggly glycine-extended gastrin₁₇
MTT 3-(4,5-dimethylthiazol-2-yl)-2,5-diphenyltetrazolium bromide
NMR Nuclear magnetic resonance

The invention includes various pharmaceutical compositions useful for ameliorating disease. The pharmaceutical compositions according to one embodiment of the invention are prepared by bringing a compound of the invention or a derivative or salt thereof, or combinations of a compound of the invention and one or more other pharmaceutically-active agents, into a form suitable for administration to a subject using carriers, excipients and additives or auxiliaries.

Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers. Preservatives include antimicrobials, anti-oxidants, chelating agents and inert gases. Other pharmaceutically acceptable carriers include aqueous solutions, non-toxic excipients, including salts, preservatives, buffers and the like, as described, for instance, in Remington's Pharmaceutical Sciences, 20th ed. Williams & Wilkins (2000) and The British National Formulary 43rd ed. (British medical Association and Royal Pharmaceutical Society of Great Britain, 2002; http://bnf.rhn.net), the contents of which are hereby incorporated by reference. The pH and exact concentration of the various components of the pharmaceutical composition are adjusted according to routine skills in the art. See Goodman and Gilman's The Pharmacological Basis for Therapeutics (7th ed., 1985).

The pharmaceutical compositions are preferably prepared and administered in dosage units. Solid dosage units include tablets, capsules and suppositories. For treatment of a subject, depending on activity of the compound, manner of administration, nature and severity of the disorder, age and body weight of the subject, different daily doses can be used. Under certain circumstances, however, higher or lower daily doses may be appropriate. The administration of the daily dose can be carried out both by single administration in the form of an individual dose unit or else several smaller dose units and also by multiple administration of subdivided doses at specific intervals.

The pharmaceutical compositions according to the invention may be administered locally or systemically in a therapeutically effective dose. Amounts effective for this use will, of course, depend on the severity of the disease and the weight and general state of the subject. Typically, dosages used in vitro may provide useful guidance in the amounts useful for in situ administration of the pharmaceutical composition, and animal models may be used to determine effective dosages for treatment of the cytotoxic side effects. Various considerations are described, eg., in Langer, Science, 249: 1527, (1990). Formulations for oral use may be in the form of hard gelatin capsules, in which the active ingredient is mixed with an inert solid diluent, for example, calcium carbonate, calcium phosphate or kaolin. They may also be in the form of soft gelatin capsules, in which the active ingredient is mixed with water or an oil medium, such as peanut oil, liquid paraffin or olive oil.

Aqueous suspensions normally contain the active materials in admixture with excipients suitable for the manufacture of aqueous suspensions. Such excipients may be suspending agents such as sodium carboxymethyl cellulose, methyl cellulose, hydroxypropylmethylcellulose, sodium alginate, polyvinylpyrrolidone, gum tragacanth and gum acacia; dispersing or wetting agents, which may be (a) a naturally occurring phosphatide such as lecithin; (b) a condensation product of an alkylene oxide with a fatty acid, for example, polyoxyethylene stearate; (c) a condensation product of ethylene oxide with a long chain aliphatic alcohol, for example, heptadecaethylenoxycetanol; (d) a condensation product of ethylene oxide with a partial ester derived from a fatty acid and hexitol such as polyoxyethylene sorbitol monooleate, or (e) a condensation product of ethylene oxide with a partial ester derived from fatty acids and hexitol anhydrides, for example polyoxyethylene sorbitan monooleate.

The pharmaceutical compositions may be in the form of a sterile injectable aqueous or oleaginous suspension. This suspension may be formulated according to known methods using suitable dispersing or wetting agents and suspending agents such as those mentioned above. The sterile injectable preparation may also a sterile injectable solution or suspension in a non-toxic parenterally-acceptable diluent or solvent, for example, as a solution in 1,3-butanediol. Among the acceptable vehicles and solvents which may be employed are water, Ringer's solution, and isotonic sodium chloride solution. In addition, sterile, fixed oils are conventionally employed as a solvent or suspending medium. For this purpose, any bland fixed oil may be employed, including synthetic mono-or diglycerides. In addition, fatty acids such as oleic acid may be used in the preparation of injectables.

Compounds of the invention may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Dosage levels of the compound of the invention will usually be of the order of about 0.5 mg to about 20 mg per kilogram body weight, with a preferred dosage range between about 0.5 mg to about 10 mg per kilogram body weight per day (from about 0.5 g to about 3 g per patient per day). The amount of active ingredient which may be combined with the carrier materials to produce a single dosage will vary, depending upon the host to be treated and the particular mode of administration. For example, a formulation intended for oral administration to humans may contain about 5 mg to 1 g of an active compound with an appropriate and convenient amount of carrier material, which may vary from about 5 to 95 percent of the total composition. Dosage unit forms will generally contain between from about 5 mg to 500 mg of active ingredient.

It will be understood, however, that the specific dose level for any particular patient will depend upon a variety of factors including the activity of the specific compound employed, the age, body weight, general health, sex, diet, time of administration, route of administration, rate of excretion, drug combination and the severity of the particular disease undergoing therapy.

In addition, some of the compounds of the invention may form solvates with water or common organic solvents. Such solvates are encompassed within the scope of the invention.

The compounds of the invention may additionally be combined with other compounds to provide an operative combination. It is intended to include any chemically compatible combination of pharmaceutically-active agents, as long as the combination does not eliminate the activity of the compound of formula I of this invention.

The invention will now be described in detail by way of reference only to the following non-limiting examples and drawings.

Materials and Methods

Chemicals and Cell Lines

Ggly and Gamide were purchased from Auspep (Melbourne, Australia) and Bachem (Bubendorf, Switzerland), respectively. Hexapeptide to nonapeptide fragments were purchased from Auspep (Melbourne, Australia). The identity of all peptides was confirmed by mass spectral analysis. The purities of the peptides, as assessed by HPLC, were: LE₅AYG, 96%; LE₅AY, 98%; E₅AY, 97%; LE₅A, 96%; LES, 97%; E₅A, 95%; E₅, 98%.

Ggly mutants were synthesized by Auspep (Melbourne, Australia). Ggly fragments were synthesized by Chiron Mimotopes (Clayton, Australia). Peptide concentrations were calculated from their absorbance at 280 nm.

The T lymphoblastoid cell line Jurkat (Galleyrand et al, 1994) was grown at 37° C. in 225-cm² culture flasks in Roswell Park Memorial Institute-1640 medium supplemented with 10% fetal bovine serum (FBS), 100 U/ml penicillin, and 100 μg/ml streptomycin.

Cos-7 cells were grown at 37° C. in 175-cm² culture flasks in Dulbecco's modified Eagle's medium (DMEM) containing 10% FBS, 100 U/ml penicillin, and 100 μg/ml streptomycin.

The IMGE-5 cell line was established from the gastric mucosa of mice transgenic for a temperature-sensitive mutant of the SV40 large T antigen (Hollande et al, 2001). IMGE-5 cells were generally grown at 33° C. in DMEM containing 1 unit/ml γ-interferon, 10% FBS, 100 U/ml penicillin, and 100 μg/ml streptomycin (permissive conditions). For all experiments, cells were transferred to 39° C. in the same medium without 7-interferon (non-permissive conditions), where they display differentiated characteristics such as expression of functional adherens and tight junctions. All experiments have been performed on cells between passages 20 and 35.

Fluorescence Spectroscopy

The tryptophan fluorescence of peptide solutions was measured in 3 mL quartz cuvettes thermostated at −25° C., with a Spex Fluorolog-2 spectrofluorimeter (Spex Industries, Edison, N.J.), with the excitation and emission wavelengths set at 290 and 345 nm, respectively. The quenching of tryptophan fluorescence induced by the binding of metal ions was used to calculate the fraction of binding sites occupied, fₐ:
fₐ=(y_{f}−y)/(y_{f}−y_{b})
where y is the fluorescence signal at a given concentration of metal ions and yb and yf are the signals when the binding sites are fully occupied and unoccupied, respectively (Winzor et al, 1995). The stoichiometry, p, and apparent dissociation constant, K_{d}, were then obtained, using the program Sigmastat (Jandel Scientific, San Rafael, Calif.), from the intercept and slope of a linear regression of the data transformed as described by Winzor and Sawyer (1995) in terms of the equation
Cₛ/fₐ=pCₐ+K_{d}/(1−fₐ)
where Cₛ is the total concentration of metal ions, and Cₐ is the total concentration of glycine-extended gastrin₁₇. For the bismuth binding studies, the data was fitted by non-linear regression with the program Sigmaplot (Jandel Scientific, San Rafael, Calif.) to the equations for models with one binding site or two binding sites with identical affinities:
fₐ=x/(K_{d1}+x)
or to the model with two binding sites with different affinities:
fₐ=(K_{d1}·x+K_{d2}·x+2x²)/2(K_{d1}·K_{d2}+K_{d1}·x+K_{d2}·x+x²)
where x is the concentration of free metal ions, K_{d1} is the first site dissociation constant and K_{d2} is the second site dissociation constant. For the one-site model the value of x at any given total concentration of metal ions was obtained by exact solution of the appropriate quadratic (Malby et al, 2001). For the two-site models the value of x was obtained by an iterative Newton-Raphson procedure (Linse et al, 1991).
Absorption Spectroscopy

Absorption spectra of peptides (100 μM in 10 mM sodium acetate (pH 4.0) containing 100 mM NaCl and 0.005% Tween 20) in the presence of increasing concentrations of ferric ions were measured against a buffer blank, in 1 ml quartz cuvettes thermostatted at 25° C., with a Cary 5 spectrophotometer (Varian, Mulgrave, Australia).

NMR Spectroscopy

For the determination of three-dimensional structure, Ggly (1.6 mM) was dissolved in 10% DMSO/10% ²H₂O/80% H₂O, and the pH was adjusted to pH 5.3 with NaO²H or ²HCl. This pH was chosen as a compromise to minimize the precipitation of gastrin that occurs at lower pH and the precipitation of ferric hydroxides at higher pH. Sequence-specific ¹H NMR resonance assignments for the trans conformation of Ggly were made from two-dimensional nuclear Overhauser enhancement spectroscopy and total correlation spectroscopy (Barnham et al, 1998, 1999). Structural constraints were then determined and structures calculated from upper bound distance constraints and backbone dihedral angle constraints based on spin-spin coupling constants. Structures were initially calculated using DYANA, refined by stimulated annealing in X-PLOR, and finally energy-minimised in X-PLOR with the CHARMM force field (Barnham et al, 1998). Structures were analyzed using MOLMOL (Version 2.1.0) (Koradi et al, 1996), and structural figures were generated using Insight II and MOLMOL.

For the subsequent experiments, samples were prepared for NMR spectroscopy by dissolving 2.5 mg of peptide in 0.6 ml of 10% DMSO/10% ²H₂O/80% H₂O at 278 K. The pH was adjusted with small amounts of 1M NaO²H or ²HCl to pH 5.3. ¹H chemical shifts were referenced to 2,2-dimethyl-2-silapenthane-5-sulfonate at 0 ppm via the chemical shift of the H₂O resonance or an impurity at 0.15 ppm. NMR spectra were recorded on Bruker DRX-600 and AMX-500 instruments, as described previously (Barnham et al, 1999).

The peptides LE₅AYG, LE₅A, E₅AY and E₅A (approximately 2 mM) were dissolved in H₂O with 5 or 10% ²H₂O. LE₅AY was slightly insoluble at 2 mM but dissolved completely on addition of ²H₆-DMSO (75% H₂O/8% ²H₂O/17% ²H₆-DMSO). The pH was adjusted to 5.3 with NaO²H/²HCl. ¹H NMR spectra were recorded at 278 K on Bruker AMX 500, Avance 500, or Avance 600 spectrometers, and referenced to 2,2-dimethyl-2-silapentane-5-sulfonate at 0 ppm via the chemical shift of the H₂O resonance at 5.00 ppm (4.96 ppm for the 17% D₆-DMSO samples). Sequence-specific ¹H NMR resonance assignments were made from two-dimensional nuclear Overhauser enhancement spectroscopy (NOESY) and total correlation spectroscopy (TOCSY).

The apparent dissociation constants, K_{d}, for the complexes between ferric ions and the nonapeptide LE₅AYG or the octapeptide LE₅AY were obtained from the change in chemical shift of the Glu10 amide proton on addition of ferric citrate using the program Sigmaplot (Jandel Scientific, San Rafael, Calif.), by linear regression of the data to the equation
1/δ=K_{d}/Δ·1/[Fe³⁺]+1/Δ
where δ is the change in chemical shift, Δ is the maximum change in chemical shift, and [Fe³⁺] is the concentration of added ferric ions.
Measurement of Inositol Phosphate Production

Intracellular inositol phosphate production was determined as described previously (Qian et al, 1993). IMGE-5 cells (10⁵ cells/ml) were plated in 24-multiwell culture plates in DMEM supplemented with 10% FBS, 200 IU/ml penicillin and 200 μg/ml streptomycin. 24 h later, cells were incubated with DMEM supplemented with antibiotics and Myo-[2-³H]inositol (2.5 μCi/well) and shifted to 39° C. for 16 h. Cells were then washed with pre-warmed DMEM and incubated (20-30 minutes, 37° C.) with DMEM supplemented with 20 mM LiCl. Loaded cells were then washed in 1 ml of IP buffer (135 mM NaCl, 20 mM N-(2-hydroxyethyl)piperazine-N′-(2-ethanesulfonic acid), 2 mM CaCl₂, 1.2 mM MgSO₄, 1 mM ethylene glycol-bis(2-aminoethylether)-N,N,N′,N′-tetraacetic acid, 10 mM LiCl, and 0.5% bovine serum albumin, pH 7.45) and incubated with or without agonist in a final volume of 0.5 ml of IP buffer. After 1-h incubation at 37° C., the reaction was stopped by removing the incubation medium and adding 1 ml of a mixture of ethanol/HCl (2000:1; v/v). One ml of each aliquot was applied to a column containing 1.6 ml of a 1:2 (v/v) Dowex AG-1-X8 anion exchange resin in distilled water. The columns were washed with 4 ml of distilled water, followed by 4 ml of 40 mM HCOONH₄, and inositol phosphates were eluted with 4 ml of 1 M HCOONH₄. The radioactivity of each eluate was counted after addition of Complete phase combining system solution (Amersham, UK). For the positive control, IMGE-5 cells were incubated with a combination of 30 mM NaF and 10 μM AlCl₃ under the same experimental conditions.

Proliferation Assay

A colorimetric assay was used to measure cell proliferation. Briefly, IMGE-5 cells were seeded in a 96-well plate at a density of 10⁴ cells/well in DMEM containing 10% FBS and antibiotics. The following day, cells were synchronized in Go by incubation for 24 h in the same medium lacking FBS. The medium was replaced the next day with fresh medium containing 1% FBS and antibiotics and the peptides under investigation, and incubation was continued for 72 h. 15 μl of 5 mg/ml 3-(4,5-dimethylthiazol-2-yl)-2-5-diphenyltetrazolium bromide (MTT, Sigma, St Louis Mo.) was added per well, and the plate was incubated at 37° C. for 4 h before the medium was discarded. 200 μl 40 mM HCl in isopropanol was added to lyse the cells, and the absorbance at 570 nm was read on a BioRad Model 550 Microplate reader (BioRad, Hercules, Calif.).

In some cases cell proliferation was also measured by [³H]-thymidine incorporation as described previously (Oiry et al., 2000). Briefly, IMGE-5 cells (10⁵/well) were seeded in 24-well plates in medium containing 10% FBS and 1 unit/ml γ-interferon. On the next day cells were serum starved and shifted to 39° C. for 24 hr. Cells were then incubated with or without various compounds at 39° C. for 17 h in medium supplemented with 0.2% bovine serum albumin only. Cells were incubated with 0.5 μCi of [³H] thymidine in the same medium at 39° C. for 4 h, washed twice with PBS (2.7 mM KCl, 142 mM NaCl, 1.5 mM KH₂PO₄, 10 mM Na₂HPO₄, pH 6.95) containing 0.2% bovine serum albumin and incubated at 4° C. for 30 min with 5% trichloroacetic acid. Finally cells were washed with 95% ethanol to remove unincorporated [³H] thymidine, and lysed with 1 M NaOH. Lysates were transferred to counting vials and neutralized with 1M HCl. The radioactivity of each lysate was counted in a β-counter after the addition of 10 ml of Pico-fluor 40 solution (Packard, Meriden, Conn.).

In some experiments IMGE-5 cells were seeded in a 96-well plate at a density of 3-5×10³ cells/well in.DMEM containing 10% FBS, and cultured at 33° C.. On the following day, the cells were serum-starved at 33° C. for 24 h. The cells were then treated with full-length or truncated Ggly at the concentrations indicated, plus or minus 1 μM DFO in DMEM containing 1% FBS and 10 μCi/ml [methyl-³H]-thymidine. The cells were cultured at 39° C. for 24 h, and then harvested using a NUNC cell harvester. The amount of ³H-thymidine incorporated through DNA synthesis was detected with a β-counter (Packard, Meriden, Conn.).

In titration experiments the effective dose required for 50% stimulation, ED50, and the maximum % stimulation, S, were then obtained, using the program Sigmaplot (Jandel Scientific, San Rafael, Calif.), by non-linear regression of the data to the equation
s=100+S·C/(ED₅₀+C)
where s is the % stimulation at a total concentration of glycine-extended gastrin₁₇ or fragment, C.
Wound Healing and Cell Migration Experiments

In order to assess the effects of different stimuli on cell migration, wound healing experiments were performed as detailed elsewhere (Hollande et al, 1997). In brief, IMGE-5 cells were grown in 12-well plates in DMEM at 33° C. until they reached 80% confluence, then shifted to 39° C. and starved of serum for 24 h. The confluent monolayer was wounded using a 20 μl-pipette tip, and cells were then washed 3 times with phosphate-buffered saline (PBS: 2.7 mM KCl, 1.5 MM KH₂PO₄, 142 mM NaCl, and 10 mM Na₂HPO₄, pH 6.95) and treated with or without different compounds in DMEM with antibiotics. Morphology and migration of cells were observed and photographed immediately, and after treatment for 17 and 24 h. Wound width was measured at six different positions on the 24 h photographs, and averages calculated.

CCK-2 Receptor Binding Assays

Jurkat cells in Roswell Park Memorial Institute (RPMI)—1640 medium were collected by centrifugation, resuspended in PBS and dispensed into glass tubes (10⁶ cells per tube) with 10⁵ cpm of [¹²⁵I]-Bolton and Hunter labelled-CCK8 ([¹²⁵I]-BH-CCK₈) with or without various unlabelled compounds at 37° C. for 45 min. Non-specific binding was evaluated with 1 μl of unlabelled CCK₈. Incubation was stopped by adding 2 ml of ice-cold PBS containing 2% bovine serum albumin. After centrifugation, supernatants were removed and the radioactivity bound to the cell membranes was measured in a γ-counter (LKB, Wallac, Finland). Estimates of IC₅₀ values, and of the levels of [¹²⁵I]-BH-CCK₈ bound in the absence of competitor, were obtained by nonlinear regression with the program Sigmaplot (Jandel Scientific, San Rafael, Calif.) to the equation
y=a/(1+x/b)
where y is the binding expressed as a percentage of the value a observed in the absence of peptide competitor, x is the concentration of peptide competitor, and b is the IC₅₀ value.
Ggly Receptor Binding Assay

Ggly was iodinated using the IODO-GEN method and purified by reversed phase high pressure liquid chromatography (Seet et al, 1987). [¹²⁵I]-Ggly binding assays were carried out by previously described methods (Yang et al, 2001). Briefly, 5×10⁵ IMGE-5 cells/well were seeded in 6-well plates and grown at 33° C. for 24 hours. The cells were then serum starved and shifted to 39° C. for 24 h. The next day they were incubated for 60 min at 39° C. in 200 mM Tris-HCl (pH 7.2) containing 100 mM KCl, 2 MM MgCl₂, 1 mM dithiothreitol, 1 mM benzamidine, 0.1% bovine serum albumin, and 5×10⁵ cpm of [¹²⁵I]Ggly with or without cold compounds. Non-specific binding was evaluated with 1 μM of cold Ggly. Cells were then washed three times in ice-cold PBS and lysed with 1 M NaOH, and the amount of radioactivity bound was measured in a γ-counter.

Statistics

Results are expressed as the means±S.E. Data were analyzed by one-way repeated measures analysis of variance. If there was a statistically significant difference in the data set, individual values were compared with the appropriate content value by Bonferroni's t-test. Differences with p values of <0.05 were considered significant.

### EXAMPLE 1

### Solution Structure of GGLY

The structure of Ggly in aqueous solution was determined by ¹H NMR spectroscopy. The presence of a proline at the third position in the Ggly sequence, which is shown in FIG. 1, allowed the peptide to adopt two conformations, cis and trans, about the Gly2-Pro3 peptide bond, as observed previously with Gamide (Torda et al., 1985). Sequence-specific ¹H NMR resonance assignments for the major (70%) trans isomer of Ggly were made from two-dimensional nuclear Overhauser enhancement spectroscopy (NOEs) and total correlation spectroscopy spectra. The chemical shifts for Ggly have been deposited in BioMagResBank (http://www.bmrb.wisc.edu) under accession number BMRB 5384.

Structures were calculated for Ggly as described above. 324 upperbound distance constraints were inferred from NOEs, which were made up of 35 intra-residue, 149 sequential, 96 medium-range (1<i−j≦4), and 43 long-range NOEs. In addition, 13 backbone dihedral angle constraints based on spin-spin coupling constants were included; no χ¹ side chain constraints for stereospecifically assigned, nondegenerate, geminal C^{βH} resonances were employed. A summary of geometric and energetic parameters for these structures is given in Table 1.

TABLE 1
Structural statistics for the 20 energy-minimized
structures of Ggly from X-PLOR.

| | |
|---|---|
| RMSD from experimental distance | 0.019 ± 0.002 |
| restraints Å (324)^{a} | |
| RMSD from experimental dihedral | 0.56 ± 0.19 |
| restraints (deg) (13)^{a} | |
| RMSD from idealized geometry | |
| Bonds (Å) | 0.0130 ± 0.0005 |
| Angles (deg) | 2.91 ± 0.08 |
| Impropers (deg) | 0.42 ± 0.03 |
| Energies (kcal mol⁻¹) | |
| E_{NOE} | 5.8 ± 1.5 |
| E_{cdih} | 0.29 ± 0.21 |
| E_{L-J} | −52.4 ± 3.5 |
| E_{bond} + E_{angle} + Eᵢₘₚᵣₒₚₑᵣ | 72.7 ± 4.8 |
| E_{elec} | −183 ± 11 |
| Mean pairwise RMSD (Å) | |
| Residue 3-7; 11-18 | 0.63 ± 0.28^{b} |
| | 1.19 ± 0.30^{c} |
| 1-18 | 1.05 ± 0.45^{b} |
| | 1.64 ± 0.42^{c} |

Structures were calculated for Ggly as described in Experimental Procedures. The backbone φ and ψ angles were 95% in the allowed region and 100% in the generously allowed region of the Ramachandran plot as determined by PROCHECK.
^{a}The numbers of restraints are shown in parentheses. None of the structures had distance violations > 0.5 Å or dihedral angle violations > 5°.
^{b}Backbone heavy atoms.
^{c}All heavy atoms.

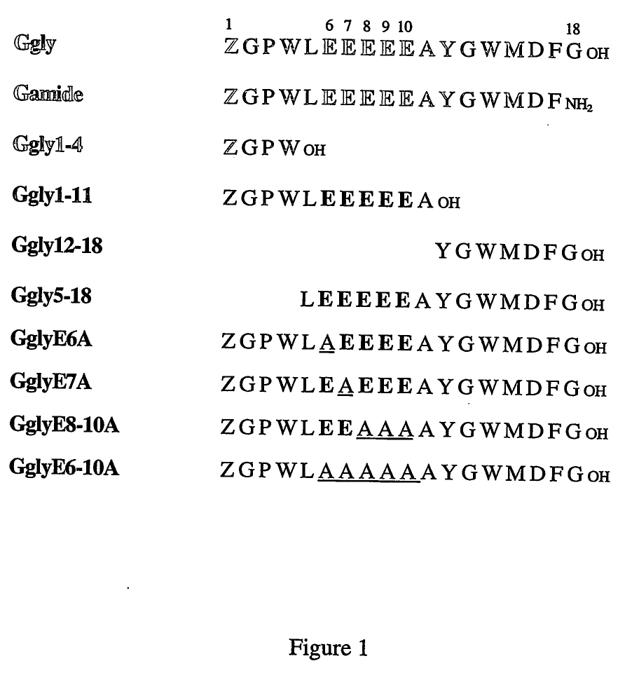

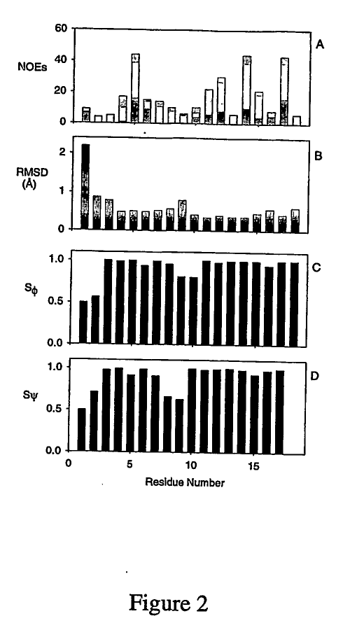

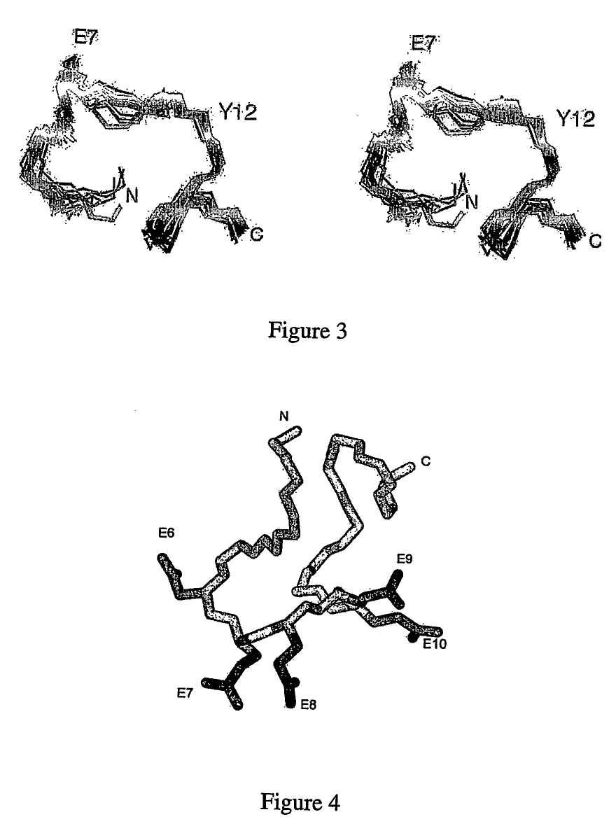

Analysis of the backbone angular order parameters (S), illustrated in FIG. 2, of the final 20 structures of Ggly, shown in FIG. 3, indicated that residues 3-7 and 11-18 were well defined, with S>0.9 for both φ and Ψ angles. The RMSD from the mean structure confirmed that most of the structure was well defined, except for the -(Glu)₅- sequence and for residues near the N terminus.

The individual Ggly structure closest to the average structure in aqueous solution was determined from two-dimensional NMR data. The overall conformation of the major (70%) trans isomer of Ggly was an amphipathic disc-like structure with hydrophobic and hydrophilic faces. This is shown in FIG. 4. Hydrophobic interactions involving residues Leu5, Tyr12, Trp14 and Phe17 stabilized a well-defined loop with no recognized secondary structural elements, apart from a β-turn near the N-terminus.

### EXAMPLE 2

### Definition of Ferric Ion Ligands

We have previously shown that Ggly bound two ferric ions in aqueous solution at pH 4.0, with an apparent K_{d} of 0.6 μM, and that the -(Glu)₅- sequence was essential for metal ion binding, as indicated by the quenching of tryptophan fluorescence of Ggly fragments by ferric ions (Baldwin et al, 2001).

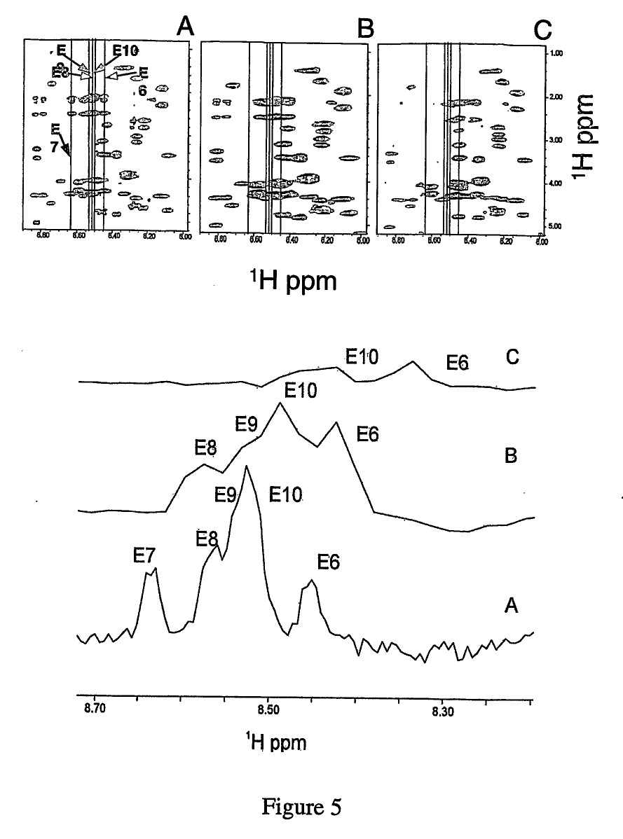

In order to define the ligands involved in ferric ion binding, we investigated the effect of ferric ions on the Ggly NMR spectrum. The results are shown in FIG. 5. As Fe(III) is paramagnetic, the resonances of any nuclei in close proximity to the metal ion will be either broadened or shifted dramatically. Addition of one equivalent of ferric ions broadened the resonances due to Glu7 beyond detection, and caused slight changes in the resonances due to Glu 8, 9 and 10, without significantly affecting other resonances. This is summarised in FIG. 5B. Similarly, as shown in FIG. 5C, addition of a second equivalent of ferric ions specifically broadened resonances due to Glu8 and 9 beyond detection. These results suggested that Glu7 was acting as one of the ligands for the first ferric ion binding site, that Glu 8 and 9 were acting as ligands for the second ferric ion binding site, and that Glu 6 and 10 were not directly involved in ferric ion binding.

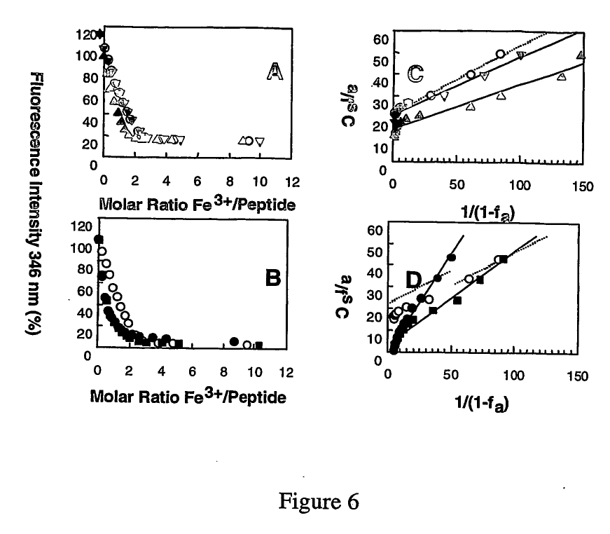

To test the hypothesis that Glu7 was a ligand in the first metal ion binding site, we examined the ability of ferric ions to quench the fluorescence of mutant Ggly peptides in which some or all of the glutamates had been replaced by alanines. Although replacement of Glu6 with Ala had little if any effect on the stoichiometry or affinity of ferric ion binding, replacement of Glu7 with Ala reduced the stoichiometry by 1 without greatly changing the apparent affinity. This is illustrated in FIGS. 6A and 6C, and in Table 2.

TABLE 2
Stoichiometry and affinity of metal ion binding by glycine-
extended gastrin17 and related peptides.

| Peptide | Stoichiometry | K_{d} (μM) | Replicates | Ref. |
|---|---|---|---|---|
| Progastrin | 2.48 ± 0.13 | 2.15 ± 0.14 | 3 | — |
| Ggly | 2.09 ± 0.07 | 0.18 ± 0.04 | 4 | — |
| Ggly1-11 | 2.01 ± 0.31 | 1.20 ± 0.04 | 3 | 8 |
| Ggly5-18 | 2.15 ± 0.18 | 0.58 ± 0.19 | 3 | 8 |
| GglyE6A | 2.20 ± 0.06 | 0.23 ± 0.02 | 5 | — |
| GglyE7A | 1.33 ± 0.16 | 0.20 ± 0.01 | 5 | — |
| GglyE8-10A | 1.23 ± 0.08 | 0.37 ± 0.07 | 5 | — |
| GglyE6-10A | 0.88 ± 0.07 | 1.20 ± 0.11 | 4 | — |

The tryptophan fluorescence of progastrin₆₋₈₀, glycine-extended gastrin₁₇ and related peptides was measured in the presence of increasing concentrations of ferric ions in 10 mM sodium acetate, pH 4.0, 100 mM NaCl, 0.005% Tween 20. Values of the stoichiometry and the apparent dissociation constant (K_{d}) were obtained from linear transformations of the data presented in FIGS. 6B and 6D by least squares fitting. Values from the indicated number of independent experiments were combined to obtain the mean values (±S.E.) presented above. No binding of ferric ions to Ggly1-4 or Ggly12-18 was observed.

The importance of Glu8 and 9 in binding the second ferric ion was confirmed by the similar reduction in stoichiometry which was observed with a peptide in which Glu8, 9 and 10 had all been replaced by alanines. This is shown in FIGS. 6B and 6D, and in Table 2. The single point mutations GglyE6A and GglyE7A did not change the structure of the peptide as assessed by two-dimensional nuclear Overhauser enhancement spectroscopy. Poor solubility prevented assessment of the structures of the GglyE8-10A and GglyE6-10A peptides by NMR.

### EXAMPLE 3

### The Role of Glutamate Residues in Biological Activity

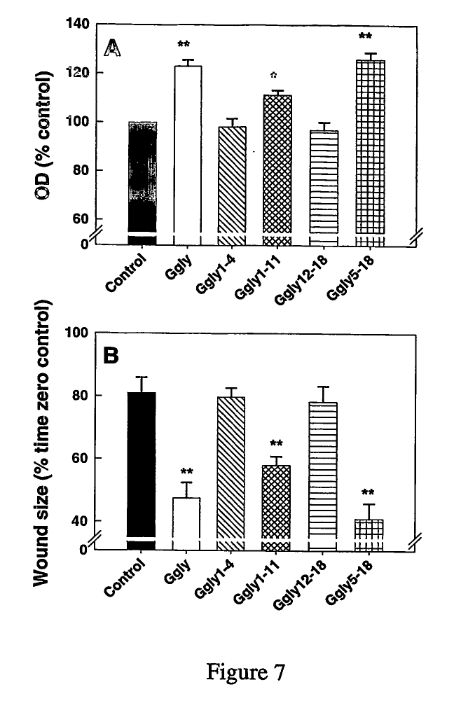

In order to identify the biologically important regions of the Ggly sequence, four Ggly fragments, Ggly1-4, Ggly1-11, Ggly12-18 and Ggly5-18, whose amino acid sequences are shown in FIG. 1, were tested for biological activity. Both Ggly1-4 and Ggly12-18 lack the -(Glu)₅- sequence, and neither of these fragments stimulated proliferation in MTT assays, as shown in FIG. 7A, or in thymidine assays (data not shown). Nor did they stimulate migration in a wound healing assay in the gastric epithelial cell line IMGE-5, as shown in FIG. 7B. In contrast, Ggly1-11, which contained the -(Glu)₅- sequence but not the C-terminal heptapeptide of Ggly, did increase proliferation, but to a significantly lesser extent than Ggly (p<0.05). Ggly5-18, which contained both the -(Glu)₅- sequence and the C-terminal heptapeptide, stimulated IMGE-5 proliferation and migration to the same extent as Ggly. These results showed that the -(Glu)₅- sequence was necessary but not sufficient for full biological activity of Ggly-like peptides, and that some or all of the C-terminal heptapeptide was also required.

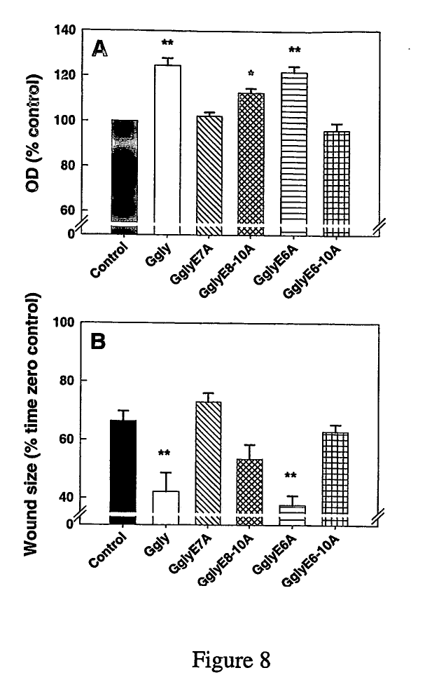

We then investigated the role of the individual glutamate residues in biological activity. The effects of four Ggly mutants in which some or all of the glutamates were replaced by alanine (see FIG. 1 for sequences) on IMGE-5 cell proliferation as assessed by MTT, as shown in FIG. 8A, and thymidine assays (data not shown), and migration as assessed in a wound healing assay, as shown in FIG. 8B, were compared. A range of concentrations from 1 pM to 1 μM was tested for each peptide, but since the potency of the active peptides was identical, results are only presented for each peptide at a concentration of 1 nM. In all cases loss of the glutamate at position 7 of Ggly resulted in loss of activity. A similar critical role for Glu7 was also noted in [³H]-thymidine proliferation assays (data not shown). The observation that GglyE7A and GglyE6-10A were inactive indicated that Glu7 played a critical role in Ggly-stimulated IMGE-5 cell proliferation and migration. However, GglyE6A stimulated cell proliferation and migration to the same extent as Ggly, and GglyE8-10A was also active in proliferation assays, although less so than Ggly (p<0.05). In contrast, Glu6, 8, 9 and 10 were not essential for activity, although substitution of one or more of Glu8, 9 and 10 reduced activity by approximately 50%. As shown above, the NMR and fluorescence data indicate that Glu7 is critical for binding the first ferric ion, and that Glu8 and Glu9 are involved in binding the second ferric ion. Thus the complete lack of activity of the GglyE7A mutant in both assays suggests that either ferric ion binding is essential for biological activity, or Glu7 is directly involved in receptor binding.

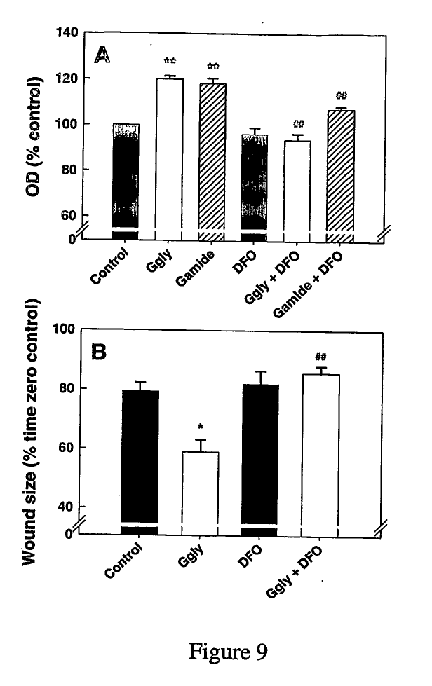

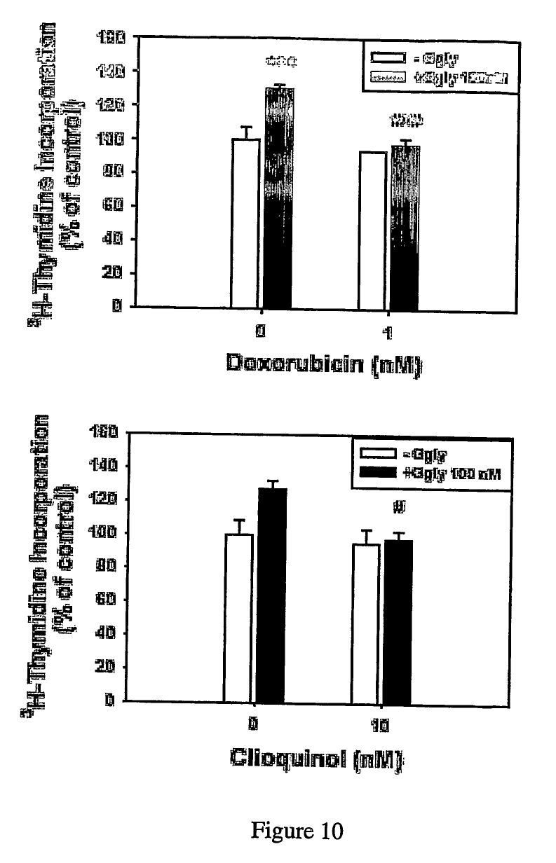

In order to distinguish between these two possibilities, we next investigated the effect of the iron chelator desferrioxamine (DFO) on the biological activity of gastrins. At a concentration which is known to have no effect on basal proliferation (Kicic et al, 2001) DFO completely blocked Ggly-stimulated IMGE-5 cell proliferation in MTT, as shown in FIG. 9A, and thymidine assays (data not shown), and migration in a wound healing assay, as shown in FIG. 9B. In contrast, DFO reduced Gamide-stimulated IMGE-5 cell proliferation by only 60% (p<0.05 V. Gamide only). Since Gamide did not stimulate IMGE-5 cell migration, the effect of DFO on this parameter could not be assessed. The effect was not restricted to DFO; other chelators such as clioquinol (FIG. 10A) or doxorubicin (FIG. 10B) also inhibited Ggly-stimulated IMGE-5 cell proliferation in a dose-dependent manner We conclude that binding of ferric ions to Glu7 is essential for the biological activity of Ggly, and may also contribute to the biological activity of Gamide.

### EXAMPLE 4

### Gastrins Bind Two Bismuth Ions

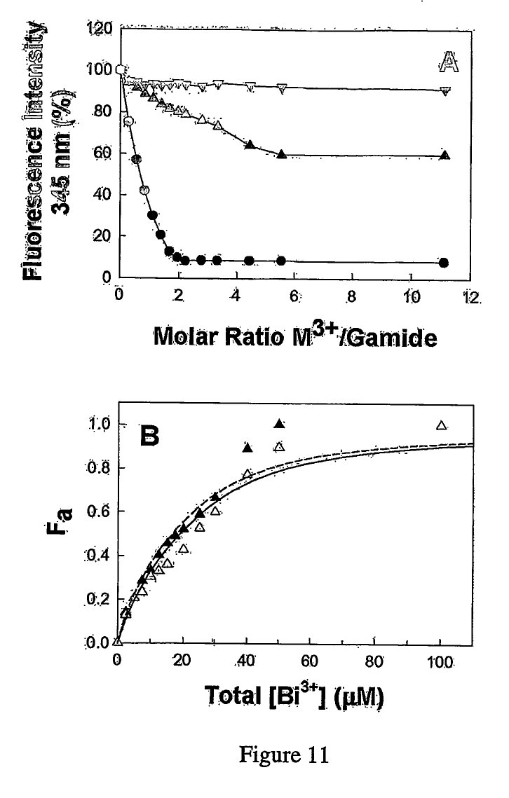

The same techniques as those described above were used to investigate the hypothesis that gastrins bind bismuth ions. We compared the ability of trivalent bismuth, chromium and ferric ions to quench the fluorescence of Gamide and Ggly. Bismuth ions quenched Gamide fluorescence, but the quenching was less marked than with ferric ions; in contrast, chromium ions had little effect on Gamide fluorescence, as shown in FIG. 11. Similar results were obtained with Ggly (data not shown). When the bismuth data were fitted by non-linear regression with the program Sigmaplot to models with either one binding site, or two binding sites with identical affinity, the two-site model illustrated in FIG. 11 gave a better fit; when a model with two non-identical binding sites was used, the K_{d} values converged. We conclude that both Gamide and Ggly bind two bismuth ions, and that the two binding sites have very similar affinities. The deviation observed between the experimental data and the lines of best fit may indicate the presence of some co-operativity between binding of the first and second bismuth ions, or of polymeric species since bismuth compounds have a propensity to polymerize. The K_{d} values obtained for the binding of bismuth ions to Gamide and Ggly were similar to each other, and were 41- and 10-fold higher than the K_{d} values for the binding of ferric ions to Gamide and Ggly, respectively, as summarized in Table 3.

TABLE 3
Affinity of Gastrins for Bismuth and Ferric Ions.

| | Bismuth | | Ferric | |
|---|---|---|---|---|
| Peptide | K_{d} (μM) | Replicates | K_{d} (μM) | Replicates |
| Gamide | 8.2 ± 0.8 | 5 | 0.2 ± 0.1 | 3 |
| Ggly | 5.8 ± 1.4 | 5 | 0.6 ± 0.2 | 12 |

The tryptophan fluorescence of Gamide and Ggly was measured in the presence of increasing concentrations of bismuth or ferric ions at pH 4.0. Values of the apparent dissociation constants (K_{d}) were obtained from data similar to that presented in FIG. 1B by nonlinear regression to the equation for a model with two identical independent binding sites with the program Sigmastat. Values from the indicated number of independent experiments were combined to obtain the mean values (±S.E.) presented above. The Kd value for the binding of ferric ions to Ggly is taken from Baldwin et al. (2001).

### EXAMPLE 5

### Glutamate Residues 7-9 Act as Bismuth Ligands

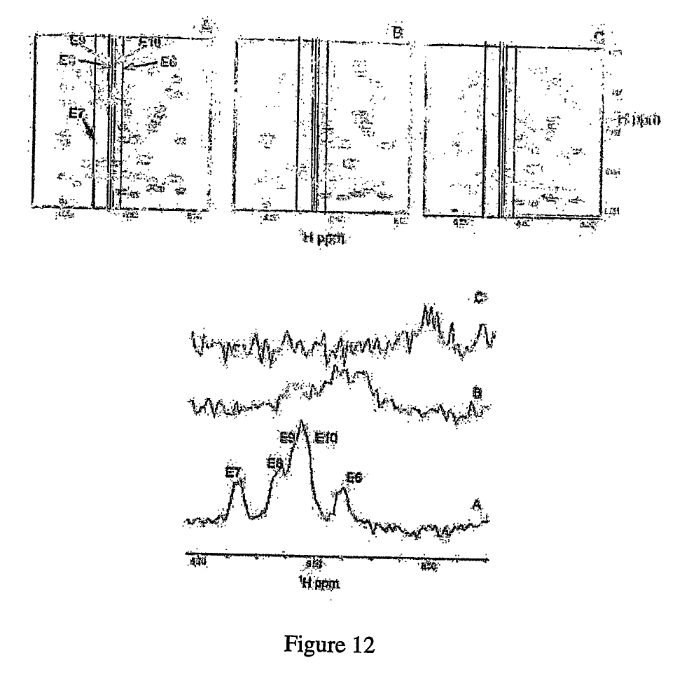

In order to define the ligands involved in bismuth ion binding, we investigated the effect of bismuth ions on the NMR spectrum of Ggly. The results are shown in FIG. 12. Addition of 4 mole of bismuth ions per mole of Ggly broadened the resonances due to Glu7, 8 and 9 beyond detection, without significantly affecting other resonances. These results suggested that the carboxyl groups of Glu7, 8 and 9 were acting as ligands for the bismuth ion, and are in agreement with our previous conclusion that Glu7 acts as a ligand for the first ferric ion binding site, and that Glu 8 and 9 act as ligands for the second ferric ion binding site.

### EXAMPLE 6

### Bismuth Ions Selectively Inhibit GGLY-Induced Inositol Phosphate Production

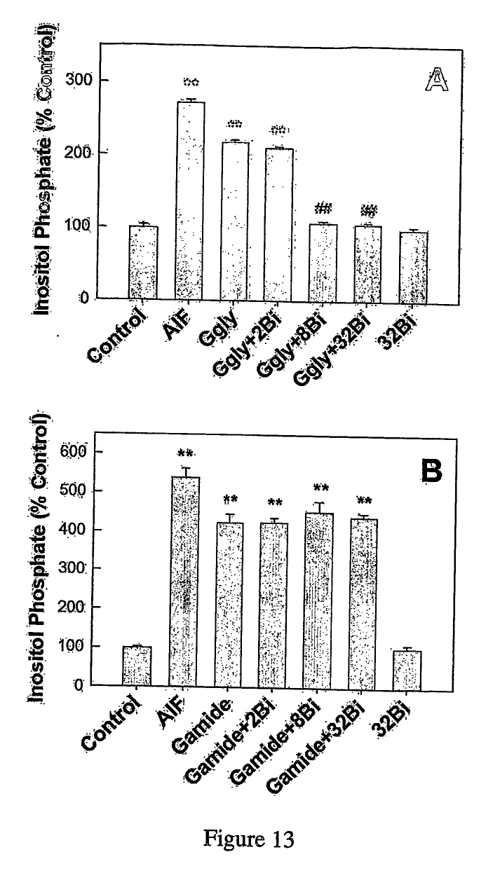

In order to assess whether the binding of bismuth ions influenced the biological activity of either Ggly or Gamide, we investigated inositol phosphate production. Ggly-stimulated inositol phosphate production was measured in HT29 human colorectal carcinoma cells. AlF₄⁻, which constitutively activates G proteins upstream of phospholipase C, induced a 2.6-fold increase in inositol phosphate production in this system, as shown in FIG. 13. Ggly significantly stimulated inositol phosphate production (225% compared to the control). In this system, Ggly is active in the absence of added ferric ions, as the Dulbecco's modified Eagle's medium contains 248 nM Fe³⁺ Addition of increasing concentrations of bismuth ions (2, 8, and 32 moles Bi³⁺/mole Ggly) significantly reduced this Ggly-induced stimulation. Similar inhibitory effects of bismuth ions on Ggly-stimulated inositol phosphate production were also observed in IMGE-5 gastric epithelial cells, but the fact that Ggly stimulation was only 110% compared to the control made quantitation unreliable.

Gamide-stimulated inositol phosphate production was measured in COS-7 cells transfected with the CCK-2 receptor. AlF₄⁻ induced a 5-fold increase in inositol phosphate production in this system, as shown in FIG. 13. Gamide also significantly stimulated inositol phosphate turnover, by a factor of 4-fold. However, in contrast to Ggly, addition of increasing concentrations of bismuth ions (2, 8, and 32 moles Bi³⁺/mole Gamide) had no significant effect on inositol phosphate production. Bismuth ions alone had no effect on inositol phosphate production in CCK-2 receptor-transfected COS-7 cells and HT29 cells, even at the highest concentration tested.

### EXAMPLE 7

### Bismuth Ions Inhibit GGLY-Induced Cell Proliferation

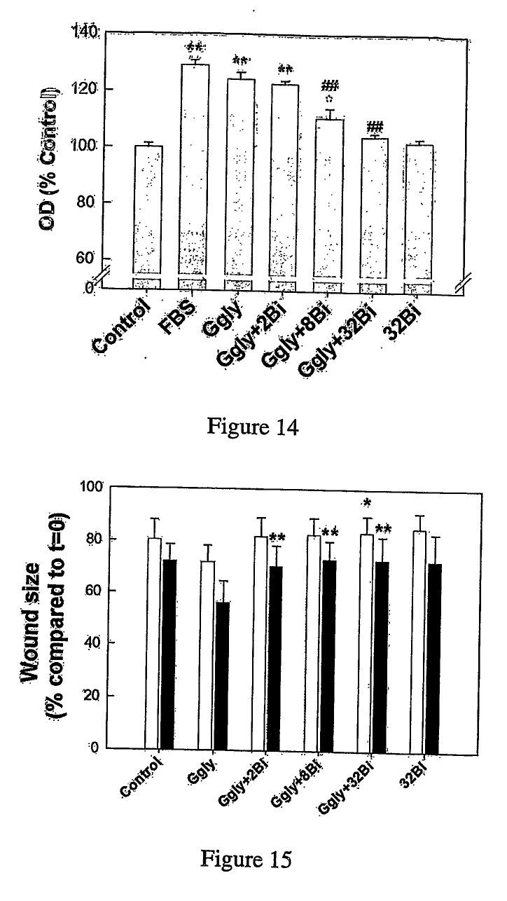

We then investigated whether bismuth ions had any effect on cell proliferation, using a MTT assay. As reported previously (Stepan et al, 1999), Ggly stimulated HT29 cell proliferation (120% compared to the control). As with inositol phosphate production, addition of increasing concentrations of bismuth ions significantly decreased the proliferation induced by Ggly. The results are shown in FIG. 14. Bismuth ions alone had no effect in this assay, even at the highest concentrations tested. Similar inhibitory effects of bismuth ions were also observed on Ggly-stimulated proliferation of the gastric carcinoma cell line AGS and the gastric epithelial cell line IMGE5, as shown in Tables 4 and 5.

TABLE 4
Effect of Bismuth on Ggly-induced Proliferation of IMGE
Cells.

| | Mean Response ± SEM | |
|---|---|---|
| Treatment | (% Control) | Significance |
| Ggly | 135.7 ± 3.6 | |
| Ggly + 2 mol/mol Bi | 121.5 ± 3.0 | NS |
| Ggly + 8 mol/mol Bi | 96.7 ± 2.1 | <0.001 |
| Ggly + 32 mol/mol Bi | 98.7 ± 5.1 | <0.001 |
| 32 mol/mol Bi | 97.9 ± 2.5 | |
| Fetal Calf serum | 163.8 ± 7.3 | |

The proliferation of IMGE gastric epithelial cells was measured with the MTT assay in the presence of 10 nM Ggly with or without increasing concentrations of bismuth ions. Fetal calf serum was included as a positive proliferative control. Values are the mean of triplicates±standard error of the mean. Statistical significance relative to the Ggly value was determined by one way analysis of variance, followed by Bonferroni's t-test.

TABLE 5
Effect of Bismuth on Ggly-induced Proliferation of AGS
Cells.

| | Mean Response ± SEM | |
|---|---|---|
| Treatment | (% Control) | Significance |
| Ggly | 126.0 ± 2.3 | |
| Ggly + 2 mol/mol Bi | 96.3 ± 3.1 | <0.001 |
| Ggly + 8 mol/mol Bi | 102.5 ± 5.4 | 0.005 |
| Ggly + 32 mol/mol Bi | 86.0 ± 4.6 | <0.001 |
| 32 mol/mol Bi | 102.2 ± 4.2 | |
| Fetal Calf serum | 127.7 ± 4.6 | |

The proliferation of AGS gastric carcinoma cells was measured with the MTT assay in the presence of 10 nM Ggly.with or without increasing concentrations of bismuth ions. Fetal calf serum was included as a positive proliferative control. Values are the mean of triplicates±standard error of the mean. Statistical significance relative to the Ggly value was determined by one way analysis of variance, followed by Bonferroni's t-test.

### EXAMPLE 8

### Bismuth Ions Inhibit GGLY-Induced Cell Megration

In order to determine whether or not the inhibition of Ggly biological activity by bismuth ions was a general phenomenon, we investigated the effect of bismuth ions on Ggly-induced cell migration. At a concentration of 2, 8 or 32 moles/mole Ggly, bismuth ions completely blocked Ggly-induced migration of IMGE-5 gastric epithelial cells as measured in wound healing assays. This is illustrated in FIG. 15. Bismuth ions alone had no effect in this assay, even at the maximum concentration tested. Gamide was not tested in this assay, because we have shown previously that this hormone had no effect on IMGE-5 cell migration (Hollande et al, 2001).

### EXAMPLE 9

### Bismuth Ions Inhibit Binding of GGLY to its Receptor

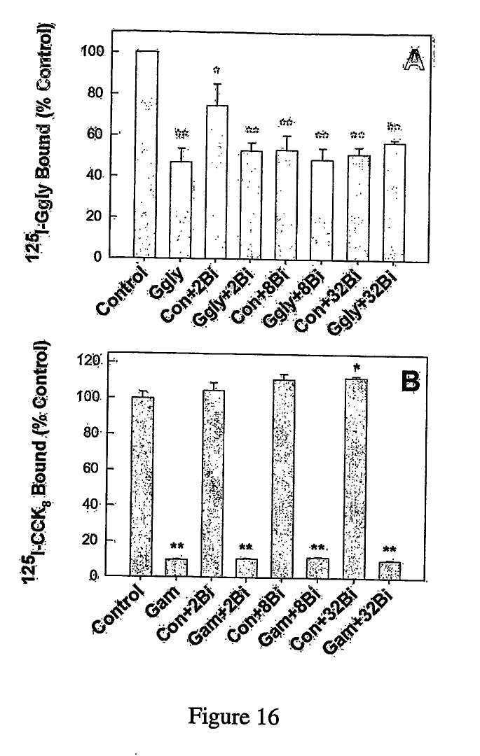

Because bismuth ions had significant inhibitory effects on inositol phosphate production, cell proliferation and migration, as shown above, we also investigated the effect of these ions on Ggly binding to IMGE-5 gastric epithelial cells. We measured the ability of 10 μM Ggly to compete with [¹²⁵I)-Ggly, with or without various concentrations of bismuth ions (2, 8, and 32 moles Bi³⁺/mole Ggly). As expected from the effects shown above on Ggly-stimulated biological activity, bismuth ions significantly inhibited the binding of [¹²⁵I)-Ggly to the Ggly receptor. Indeed, even at concentration as low as 2 moles Bi³⁺/mole Ggly inhibited [¹²⁵I]-Ggly binding by 50%, and complete inhibition was observed in the presence of 8 or 32 moles Bi³⁺/mole Ggly, as shown in FIG. 16. Although bismuth ions significantly inhibited binding of Ggly to the Ggly receptor, they had no effect on binding of CCK8 to the CCK-2 receptor.

### EXAMPLE 10

### Bismuth Ions have Little Effect on Binding of Gamide to the CCK-2 Receptor

The absence of a major inhibitory effect of bismuth ions on the biological activities of Gamide suggested that bismuth ions would not significantly inhibit the binding of Gamide to the CCK-2 receptor. In order to test this hypothesis, we measured the effect of bismuth ions on the ability of increasing concentrations of Gamide to compete with [¹²⁵I]-BH-CCK₈ for binding to CCK-2 receptors on the T-lymphoblastoid cell line Jurkat. The results are summarized in FIG. 16. On addition of 8 moles Bi³⁺/mole Gamide, the only change observed was a slight but significant increase in IC₅₀ value, from 4.8±1.3 nM to 10.0±2.7 nM (p=0.025).

### EXAMPLE 11

### Specificity and Stability of the Interaction between Glycine-Extended Gastrins and Metal Ions

Glycine-extended gastrin₁₇ (Ggly) binds 2 ferric or bismuth ions with high affinity in aqueous solution. We have previously found that Ggly does not bind Co(II), Cu(II), Mn(II), or Cr(III) ions, as detected by measurement of Ggly fluorescence in the presence of added metal ions (Baldwin et al, 2001).

In order to determine the specificity of the interaction between progastrin and metal ions, the binding of metal ions to recombinant human progastrin₆₋₈₀ was determined by measuring their ability to compete with radioactive ⁵⁹Fe³⁺ for binding to the protein by the following methods (Baldwin et al, 2001).

Progastrin₆₋₈₀ (5.6 μM) in pH 2.8 buffer (10 mM sodium formate, pH 2.8, 100 mM NaCl, 0.005% Tween 20) was incubated for 30 min at 4° C. with ⁵⁹Fe³⁺ ions (10 μM, 23.6 cpm/pmol) in the presence of several di- and trivalent metal ions (100 μM). Excess radioactivity was removed by overnight dialysis (SpectraPor dialysis tubing, molecular weight cut-off 3,500, Spectrum Labs., Rancho Dominguez, Calif.) at 4° C. against pH 4.0 buffer (10 mM sodium acetate, pH 4.0, 100 mM NaCl, 0.005% Tween 20), containing 1 gM EDTA. The radioactivity associated with progastrin₆₋₈₀ was determined by counting aliquots of the internal and external solutions in a γ-counter (LKB-Wallac, Turku, Finland).

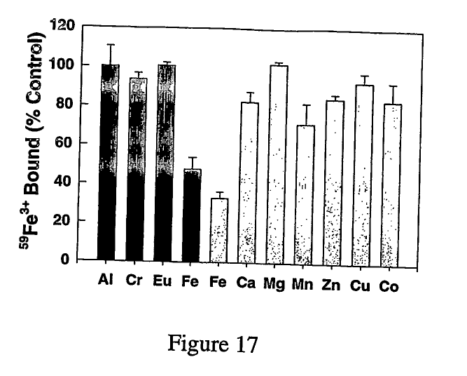

The results are shown in FIG. 17. Comparison of the ability of several divalent (grey bars) and trivalent (black bars) metal ions to compete with ⁵⁹Fe³⁺ for binding to recombinant human progastrin₆₋₈₀ indicated that only ferric and ferrous ions were able to compete effectively. We conclude that the polyglutamate binding site common to both progastrin₆₋₈₀, Ggly, and other non-amidated gastrins is highly selective in its recognition of metal ions.

### EXAMPLE 12

### Stability of the Progastin-Ferric Ion Complex

In order to determine the stability of the complex between progastrin and ferric ions, the complex was prepared by mixing recombinant human progastrin₆₋₈₀ with a 4-fold excess of radioactive ⁵⁹Fe³⁺ at pH 2.8. After removal of excess ⁵⁹Fe³⁺ ions by dialysis at low pH, the stability of the complex was determined by measuring the residual radioactivity during dialysis against buffers of different pH containing 1 μM EDTA. The data sets were well fitted by an exponential decay model, and the half lives determined under a variety of conditions are given in Table 6. We conclude that the complex is very stable under physiological conditions, with a half-life of 36 hours at pH 7.6 and 37° C.

The complex between ⁵⁹Fe³⁺ ions (20 μM) and recombinant human progastrin₆₋₈₀ (5 μM) was prepared in 10 mM sodium formate, pH 2.8, 100 mM NaCl, 0.005% Tween 20, and excess ferric ions were removed by overnight dialysis at 4° C. against 10 mM sodium acetate, pH 4.0, 100 mM NaCl, 0.005% Tween 20 or against 10 mM sodium MOPS, pH 7.6, 100 mM NaCl, 0.005% Tween 20, each containing 1 μM EDTA. Aliquots of the complex were then dialysed against fresh samples of the same buffers pre-equilibrated at 4° C., 25° C., or 37° C.. At various times duplicate samples of the external solution were counted in a γ-counter, and the radioactivity (R) associated with progastrin₆₋₈₀ at time t was expressed as a percentage of the total radioactivity (R_{T}) inside and outside the dialysis tubing at the end of the experiment, after correction for radioactive decay. Estimates of half lives (t_{0.5}) were obtained by least squares fitting of the linearised data to the equation for exponential decay
ln(R/R_{T})=−0.693 t/t_{0.5}

with the program Sigmaplot. The half lives determined in at least 3 independent experiments were used to calculate the mean values and standard errors presented in Table 6.

TABLE 6
Stability of the Ferric Ion/Progastrin₆₋₈₀ Complex.

| | |
|---|---|
| Temperature | Half Life (hours) |

| (° C.) | pH 4.0 | pH 7.6 |
|---|---|---|
| 4 | 53 ± 9 | 125 ± 27 |
| 25 | 16 ± 3 | 2800 ± 190 |
| 37 | 4.7 ± 1.0 | 36 ± 8 |

### EXAMPLE 13

### Requirement for Ferric Ions for the Biological Activity of GGLY in Normal Rectal Mucosa In Vivo

We have demonstrated in Example 3 that ferric ions are essential for the biological activity of Ggly in vitro. Thus mutation of the glutamate residue which acts as ligand at the first ferric ion binding site to alanine (GglyE7A), or treatment with the iron chelator desferrioximine (DFO) or with bismuth ions, abolishes Ggly activity. We have previously shown that Ggly stimulates proliferation of the hypoplastic rectal mucosa in rats with surgically defunctioned rectums (Aly et al, 2001). To test whether or not ferric ions are essential for the biological activity of Ggly in vivo, the effects of the GglyE7A mutant and Ggly on rectal proliferation were compared in this animal model. The effect of treatment with DFO or bismuth ions on Ggly stimulation was also observed.

Rats were divided into the following 7 treatment groups, with a maximum of 10 rats per group, and rectally defunctioned as described previously:
(a) Control: saline infusion,
(b) Control: DFO injection,
(c) Control: bismuth gavage,
(d) Ggly infusion,
(e) Ggly infusion+DFO injection,
(f) Ggly infusion+bismuth gavage, and
(g) GglyE7A infusion.

In our previous study groups of 10 rats were sufficient for the observation of statistically significant differences (Aly et al, 2001).

Ggly, or the GglyE7A mutant, was infused intraperitoneally via Alzet osmotic minipumps at a rate of 2.5 nmol/kg/hr. DFO (400 mg/kg) was administered intraperitoneally thrice weekly, and bismuth ions were administered as colloidal bismuth subcitrate (135 mg/kg/12 hours) by oral gavage 5 days per week. After 4 weeks, the rats were injected with vincristine to arrest cycling cells in metaphase, and sacrificed 3 hrs later. Crypt heights in the rectal mucosa, and the number of metaphase arrested cells per crypt, were then determined microscopically.

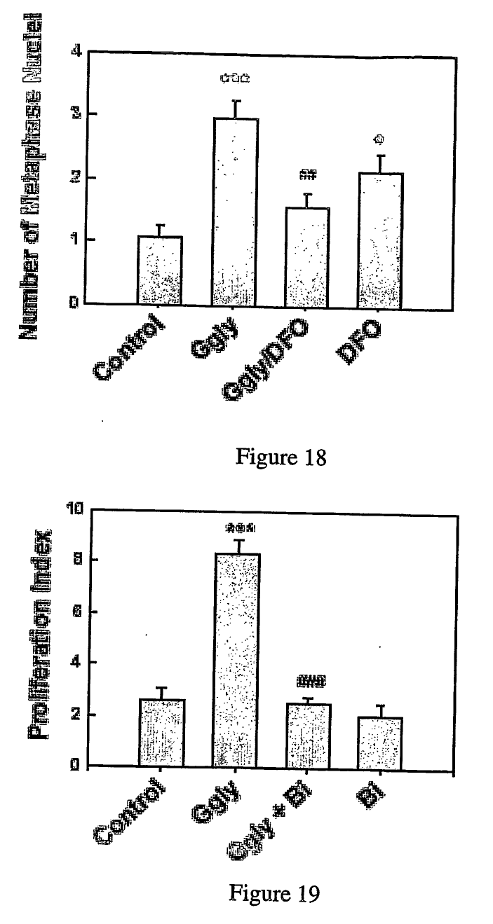

The results are shown in FIGS. 18 and 19. Treatment of rectally defunctioned rats with DFO significantly reduced the stimulation of proliferation of the rectal mucosa by Ggly (FIG. 18). Treatment of rectally defunctioned rats with bismuth ions completely abolished the stimulation of proliferation of the rectal mucosa by Ggly (FIG. 19). Contrary to our expectation prior to performing the experiment, the mutant GglyE7A was able to stimulate mucosal proliferation as well as Ggly in this model (FIG. 18), although it was unable to stimulate cell proliferation or migration in vitro (FIG. 8). This difference is probably caused by the longer time scale of the in vivo experiments (1 month v. 3 days), during which time Ggly may be broken down to shorter peptides. The data presented in Example 16 indicate that fragments such as LEEEEEAYG are still fully active, but bind ferric ions via E9 and E10. Hence the corresponding fragment derived from GglyE7A (LEAEEEAYG) would still bind ferric ions, and would therefore be fully active.

### EXAMPLE 14

### Requirement for Ferric Ions for the Stimulation of Intestinal Polyp Development by GGLY In Vivo

To test whether or not ferric ions are essential for the stimulation of intestinal polyp development by Ggly in vivo, the effect of treatment with DFO or bismuth ions on Ggly stimulation is observed in Min^{+/-} mice, which carry a dominant mutation in the APC gene, and spontaneously develop large numbers of intestinal polyps (Moser et al., 1990). The observation that the number of polyps was significantly increased in Min^{+/-} mice which had been crossed with mice overexpressing Ggly, and decreased in Min^{+/-} mice which had been crossed with gastrin-deficient mice (Koh et al., 2000), suggests that short term infusion of Ggly is likely to increase polyp numbers in this model. This model, and the models described in the two following examples, are chosen to represent three different stages in the progression from poylp to colorectal carcinoma (CRC).

Min^{±} mice are divided into 6 treatment groups, with at least 10 animals per group, and treated as follows:
1. Control, saline infusion+saline injection,
2. Control, saline infusion+DFO injection,
3. Control, saline infusion+bismuth gavage,
4. Ggly infusion+saline injection,
5. Ggly infusion+DFO injection,
6. Ggly infusion+bismuth gavage.

Ggly or saline are administered via 200 μl Alzet osmotic mini-pumps implanted intraperitoneally. Both Ggly- and saline-infused animals are treated with DFO by intraperitoneal injections (400 mg/kg) thrice weekly, and with bismuth ions by oral gavage (135 mg/kg/12 hours) 5 days per week. After 4 weeks the animals are sacrificed, and the numbers of small intestinal polyps determined microscopically. We expect that treatment of Min^{+/-} mice with DFO or bismuth ions will abrogate the previously observed increase in polyp numbers in animals overexpressing Ggly.

### EXAMPLE 15

### Requirement for Ferric Ions for the Stimulation of Tumour Induction by GGLY In Vivo

To test whether or not ferric ions are essential for the stimulation of tumour induction by Ggly in vivo, the effect of treatment with DFO or bismuth ions on Ggly stimulation is observed in rats treated with the colon-specific carcinogen azoxymethane (AOM). Colonic tumours resulting from treatment with AOM closely resemble human CRC in morphology and development (Mclellan & Bird, 1988). Progastrin-derived peptides such as Ggly have already been shown in our laboratory to stimulate the development of chemically-induced CRC in this assay (Aly et al., 2001).

Sprague-Dawley rats are divided into 6 treatment groups, with at least 10 animals per group, and treated as follows:
1. Control, saline infusion+saline injection,
2. Control, saline infusion+DFO injection,
3. Control, saline infusion+bismuth gavage,
4. Ggly infusion+saline injection,
5. Ggly infusion+DFO injection,
6. Ggly infusion+bismuth gavage.

Rats are injected weekly for 2 weeks with AOM (15 mg/kg). The effect of Ggly on tumour development is assessed by implanting 2 ml Alzet osmotic mini-pumps containing Ggly or saline into the peritoneum. Both Ggly- and saline-infused animals are treated with DFO by intraperitoneal injections (400 mg/kg) thrice weekly, or with bismuth ions by oral gavage (135 mg/kg/12 hours) 5 days per week. After 4 weeks the numbers of aberrant crypt foci are determined by microscopic examination of the colonic mucosa after staining with methylene blue. Although ethical considerations will limit study of these animals to one month, Singh and coworkers have recently shown that in mice overexpressing progastrin, the elevated numbers of aberrant crypt foci after treatment with AOM ultimately result in increased numbers of colorectal carcinomas (Singh et al., 2000b). We expect that treatment of AOM-injected rats with DFO or bismuth ions will abrogate the previously observed stimulation of tumour development by Ggly (Aly et al., 2001).

### EXAMPLE 16

### Requirement for Ferric Ions for the Stimulation of Tumour Xenograft Growth by GGLY In Vivo

To test whether or not ferric ions are essential for the stimulation of tumour xenograft growth by Ggly in vivo, the effect of treatment with DFO or bismuth ions on Ggly stimulation is observed.

Nude mice are divided into 6 treatment groups, with at least 10 animals per group, and injected subcutaneously in the underside of the flank with 3-5 million CRC cells in 0.1 ml of phosphate buffered saline. The human CRC cell line DLD-1 is chosen because Ggly has been shown to stimulate growth (Litvak et al., 1999); other CRC cell lines are also tested. The following treatments commence once tumours reach a volume of approximately 0.1 cm³:
1. Control, saline infusion+saline injection,
2. Control, saline infusion+DFO injection,
3. Control, saline infusion+bismuth injection,
4. Ggly infusion+saline injection,
5. Ggly infusion+DFO injection,
6. Ggly infusion+bismuth injection.

Ggly is infused intraperitoneally at a rate of 2.5 nmol/kg/hr via Alzet osmotic minipumps implanted subcutaneously between the shoulder blades. DFO (400 mg/kg) is administered intraperitoneally thrice weekly. Bismuth ions (135 mg/kg) are administered as colloidal bismuth subcitrate by oral gavage twice daily, 5 days per week. Tumour volumes are measured 3-5 times per week with standard calipers. After 4 weeks mice are given an intraperitoneal injection of bromodeoxyuridine (50 mg/kg), anaesthetised 1 hour later and sacrificed. Xenografts and samples of colonic mucosa are collected, and the incorporation of bromodeoxyuridine into new DNA measured by immunohistochemistry. We expect that treatment of nude mice with DFO or bismuth ions will abrogate the previously observed stimulation of proliferation of DLD-1 xenografts by Ggly (Litvak et al., 1999).

### EXAMPLE 17

### Preparation of Cr(III)GGLY and Co(III)GGLY

The rates at which metal ion ligands can be exchanged vary greatly, depending on the metal ion and on its oxidation state. Thus metal ions such as Cr(II), whose ligands exchange rapidly, are termed exchange-labile, while metal ions such as Cr(III), whose ligands exchange slowly, are termed exchange-inert. The advantage of an exchange-inert complex is that it will not dissociate on dilution, and will therefore be stable when injected into the body or when taken orally. In this example exchange-inert Cr(III) and Co(III) complexes of Ggly are prepared, and their ability to compete with Fe(III)Ggly for binding to Ggly receptors is tested.

The Co(II) complex of Ggly is prepared by addition of 2 equivalents of CoCl₂ to a solution of Ggly. The Co(II) is then oxidised in situ to Co(III) by treatment with hydrogen peroxide in the presence of phenol as a free radical scavenger (Anderson and Vallee, 1977). Cr(II)Cl₂ is prepared by reduction of Cr(III)Cl₂ with zinc amalgam in 0.1 M HCl (2). The Cr(III)Ggly complex is prepared by the method of Balakhrishnan and Villafranca (1979). The Cr(II) complex of Ggly is then prepared by addition of Ggly to a solution containing 2 equivalents of Cr(II)Cl₂ under strictly anaerobic conditions). Oxidation to the Cr(III)Ggly complex is achieved by subsequent exposure to atmospheric oxygen. The Co(III)Ggly and Cr(III)Ggly complexes are purified by reverse phase HPLC, and tested for their ability to block the ability of Ggly to stimulate proliferation and migration of the IMGE-5 gastric epithelial cell line, using the method described herein.

Since the Bi(III)Ggly complex is able to block the ability of Ggly to stimulate proliferation and migration of the IMGE-5 gastric epithelial cell line, as shown in Examples 7 and 8, we expect that the Co(III)Ggly and Cr(III)Ggly complexes will also act as antagonists of the Ggly receptor. The slow rate of metal ion dissociation from these complexes suggests that they may act as long-lasting antagonists of the Ggly receptor in vivo.

### EXAMPLE 18

### The Role of Ferric Ions and Glutamates in the Biological Activity of Gamide

Definition of Ferric Ion Ligands

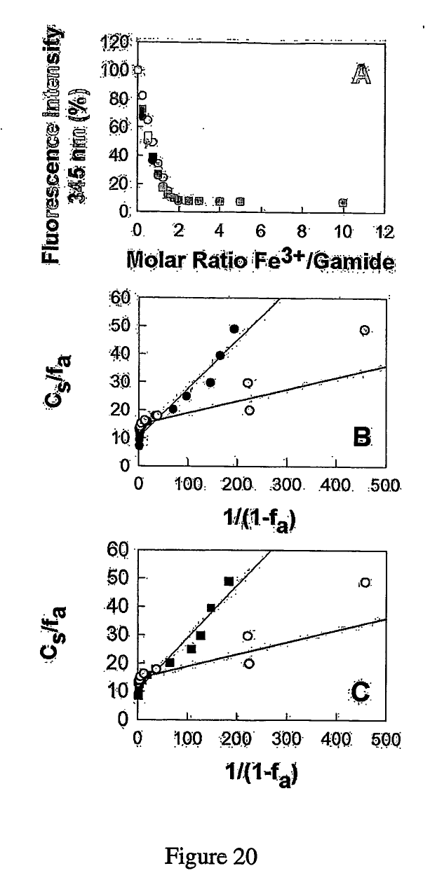

In order to confirm that Glu7-9 were also involved in ferric ion binding to Gamide, we first investigated the ability of ferric ions to quench the fluorescence of mutant Gamide peptides in which Glu7, or Glu8 and Glu9, had been replaced by alanines. As observed with Ggly in Example 2, replacement of Glu7 with Ala reduced the stoichiometry of ferric ion binding by 1 without greatly changing the apparent affinity, as illustrated in FIG. 20. Values of the stoichiometry and the apparent dissociation constant were obtained from the intercept and slope, respectively, of linear transformations of the data for peptides with single (B) or double (C) alanine substitutions by least squares fitting with the program Sigmastat. The values of at least 3 independent experiments were combined to obtain the mean values presented in Table 7. Substitution of the glutamate(s) at position 7, or at positions 8 and 9, of Gamide resulted in a reduction of one in the number of bound ferric ions.

TABLE 7
Stoichiometry and affinity of ferric ion binding by
amidated gastrin17 and related peptides.

| Peptide | Stoichiometry | K_{d} (μM) | Replicates |
|---|---|---|---|
| Gamide | 1.56 ± 0.09 | 0.20 ± 0.14 | 3 |
| GamideE7A | 0.87 ± 0.04 | 0.17 ± 0.03 | 4 |
| GamideE8, 9A | 1.12 ± 0.11 | 0.16 ± 0.06 | 4 |

The tryptophan fluorescence of amidated gastrin₁₇ and related peptides was measured in the presence of increasing concentrations of ferric ions at pH 4.0 (8). Values of the stoichiometry and the apparent dissociation constant (K_{d}) were obtained from linear transformations of the data presented in FIG. 20A by least squares fitting. Values from the indicated number of independent experiments were combined to obtain the mean values (±S.E.) presented above.

The importance of Glu8 and 9 in binding the second ferric ion was confirmed by the similar reduction in stoichiometry observed with a mutant Gamide peptide in which Glu8 and 9 had both been replaced by alanines. Role of Glutamates in CCK-2 Receptor Binding In order to determine the importance of Glu7-9 in the binding of Gamide to the CCK-2 receptor, the two mutant peptides GamideE7A and Gamide E8,9A were tested for their ability to bind to COS-7 cells transiently transfected with CCK-2 receptor cDNA. The ability of increasing concentrations of Gamide (A), GamideE7A (B), and Gamide E8,9A (C) (Auspep, Melbourne, Australia) to compete with [¹²⁵I]-BH-CCK₈ (150 pM, 100,000 cpm) for binding to the human CCK-2 receptor on transiently transfected COS-7 cells was measured.

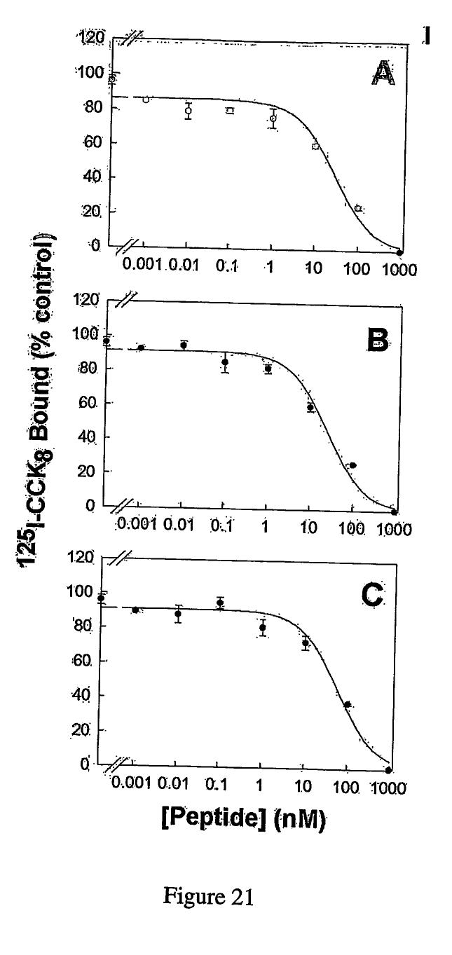

Both peptides retained the ability to compete with [¹²⁵I]-BH-CCK₈, but their affinities for the CCK-2 receptor were slightly, but not significantly, lower than the affinity for Gamide. This is illustrated in FIG. 21A. The lines of best fit shown in FIG. 21 were obtained by nonlinear regression to a single site model with the program Sigmaplot. The values for IC₅₀ and for the predicted ordinate intercept were as follows: Gamide, 28.9 nM, 86.1%; GamideE7A, 25.1 nM, 91.6%; GamideE8,9A, 58.2 nM, 91.0%. These values were combined with the data from at least two other experiments to obtain the mean values presented in Table 8. Substitution of the glutamates at position 7, or at positions 8 and 9, of Gamide did not significantly reduce the affinity of binding to the human CCK-2 receptor on transiently transfected COS-7 cells.

TABLE 8
Affinity of amidated gastrin17 and related peptides for the CCK-2 receptor.

| | |
|---|---|
| COS-7 | Jurkat |

| | % Maximum | | | | % Maximum | | | |
|---|---|---|---|---|---|---|---|---|
| Peptide | Binding | IC₅₀ (nM) | Replicates | p | Binding | IC₅₀ (nM) | Replicates | p |
| Gamide | 94.2 ± 2.4 | 27 ± 7 | 5 | | 100.0 ± 3.1 | 1.3 ± 0.2 | 6 | |
| GamideE7A | 92.0 ± 1.0 | 37 ± 11 | 5 | NS | 96.7 ± 4.6 | 3.1 ± 0.2 | 3 | 0.005 |
| GamideE8, 9A | 98.4 ± 2.1 | 112 ± 39 | 5 | NS | 97.8 ± 9.0 | 2.8 ± 0.5 | 3 | 0.017 |

Binding of ¹²⁵I-BH labelled-CCK₈ to COS-7 cells transiently transfected with the CCK-2 receptor, or to Jurkat cells, was measured in displacement experiments with increasing concentrations of Gamide or related peptides as described for FIG. 21.
Values of the % maximum binding in the absence of competitor and of the IC₅₀ were obtained from the data presented in FIG. 21 by least squares fitting with the program Sigmaplot.
Values from the indicated number of independent experiments were combined to obtain the mean values (±S.E.) presented above.
Data were analyzed by one-way analysis of variance. If there was a statistically significant difference in the data set, individual values were compared with the Gamide value by Bonferroni's t-test.

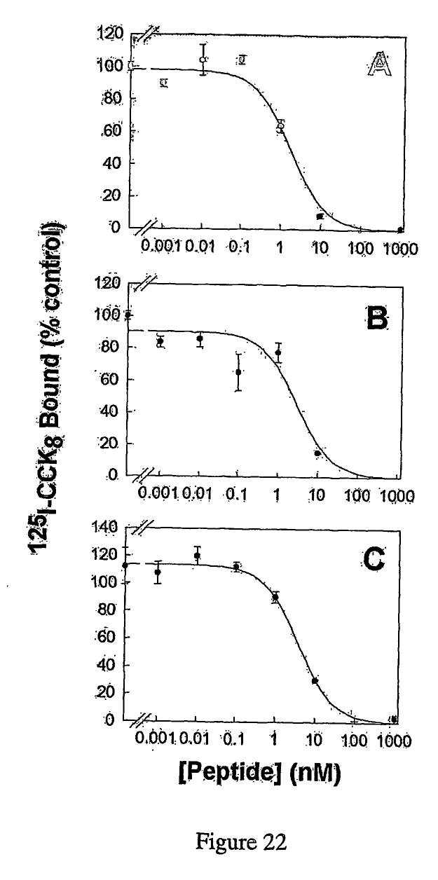

Because the affinity for Gamide was itself higher than previously reported values obtained with COS-7 cells (IC₅₀=0.94 nM (6); IC₅₀=6.4 nM (25)), the same experiments were repeated with the T lymphoblastoid cell line Jurkat, which has been reported to express CCK-2 receptors (IC₅₀=1.05 nM (21)). The affinities of all peptides for the CCK-2 receptor on Jurkat cells were approximately 10- to 20-fold lower than for the receptor on COS-7 cells, as shown in FIG. 22. As before, the affinities of the mutant peptides for the CCK-2 receptor were slightly lower than the affinity for Gamide, but the difference was significant for GamideE7A only.

Role of Glutamates in Biological Activity

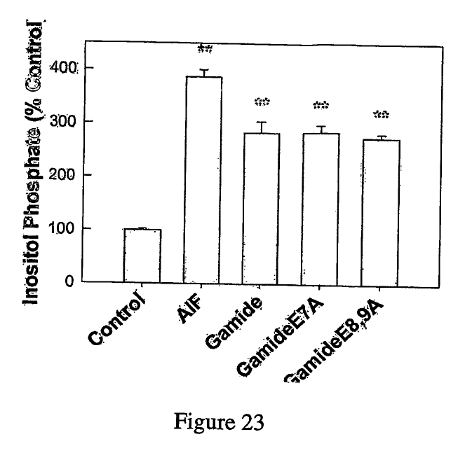

We then investigated the role of the individual glutamates in biological activity. The effects of the mutants GamideE7A and GamideE8,9A on inositol phosphate production in COS-7 cells transiently transfected with the CCK-2 receptor were compared. The effect of 10 μM AlF₄⁻ (AlF), or 10 nM Gamide, GamideE7A, or GamideE8,9A (Auspep, Melbourne, Australia) on inositol phosphate production in COS-7 cells transiently transfected with the human CCK-2 receptor was measured. No significant difference was observed in the extent of stimulation by Gamide, GamideE7A, or GamideE8,9A. Similar results were obtained in three independent experiments. The results are shown in FIG. 23.

The observation that GamideE7A and GamideE8,9A were both active indicated that Glu7 did not play a critical role in Gamide-stimulated inositol phosphate production.

Role of Ferric Ions in CCK-2 Receptor Binding and Biological Activity

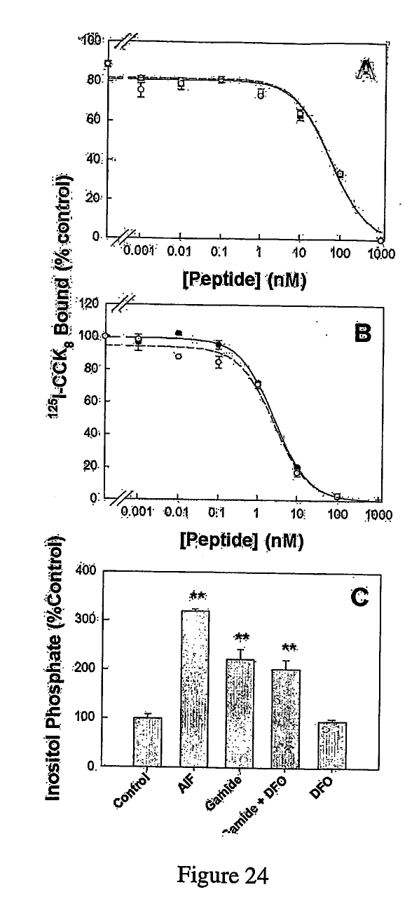

In order to confirm that the binding of ferric ions to Glu7-9 did not affect the binding of Gamide to the CCK-2 receptor, the ability of the iron chelating agent desferrioxamine (DFO) to interfere with the binding of ([¹²⁵I])-BH-CCK₈) to the CCK-2 receptor was investigated. As shown in FIG. 24, no significant difference in the IC₅₀ value for Gamide binding to the CCK-2 receptor in transiently transfected COS cells (FIG. 24A) or in Jurkat cells (FIG. 24B) was observed in either the presence or absence of DFO.

We also investigated the effect of DFO on the biological activity of gastrins. DFO had no effect on Gamide-stimulated inositol phosphate production in COS-7 cells transiently transfected with the CCK-2 receptor as shown in FIG. 24B. The values shown in FIG. 24 were combined with the data from at least two other experiments to obtain the mean values presented in Table 9.

TABLE 9
Effect of DFO on the affinity of amidated gastrin17 for the CCK-2 receptor.

| | |
|---|---|
| COS-7 | Jurkat |

| | % Maximum | | | | % Maximum | | | |
|---|---|---|---|---|---|---|---|---|
| Peptide | Binding | IC₅₀ (nM) | Replicates | p | Binding | IC₅₀ (nM) | Replicates | p |
| Gamide | 81.1 ± 1.7 | 57 ± 11 | 1 | | 99.0 ± 0.4 | 3.3 ± 0.8 | 3 | |
| Gamide + DFO | 81.7 ± 1.6 | 58 ± 11 | 1 | NS | 96.0 ± 1.2 | 3.6 ± 0.9 | 3 | NS |

Binding of ¹²⁵I-BH-labelled-CCK₈ to COS-7 cells transiently transfected with the CCK-2 receptor, or to Jurkat cells, was measured in displacement experiments with increasing concentrations of Gamide in the presence and absence of 1 μM DFO as described in the FIG. 7 legend.
Values of the % maximum binding in the absence of competitor and of the IC₅₀ were obtained from the data presented in FIG. 7 by least squares fitting with the program Sigmaplot.
Values from the indicated number of independent experiments were combined to obtain the mean values (±S.E.) presented above.
Data were analyzed by Student's t-test.

We conclude that binding of ferric ions to Glu7-9 is not essential for the biological activity of Gamide.

### EXAMPLE 19

### Gastrin Fragments also Possess Metal-Ion Dependent Activity

In order to define the minimum biologically active fragment of Ggly, and to determine whether ferric ions were required for its activity, we investigated the activities of Ggly fragments containing the five glutamate residues (LE₅AYG, LE₅AY, E₅AY, LE₅A, LE₅, E₅A and E₅) in proliferation assays. The stoichiometry of ferric ion binding to the Ggly fragments containing tyrosine was determined by absorption spectroscopy, and the iron ligands were defined by NMR spectroscopy.

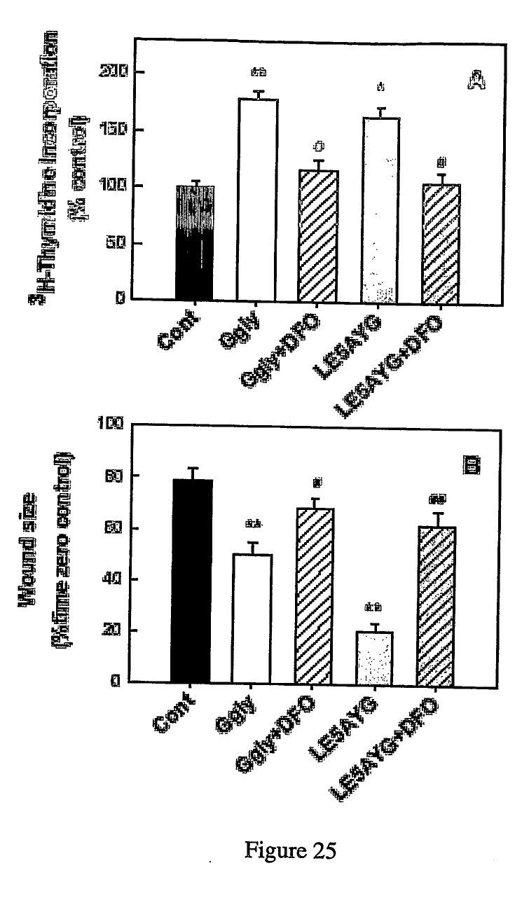

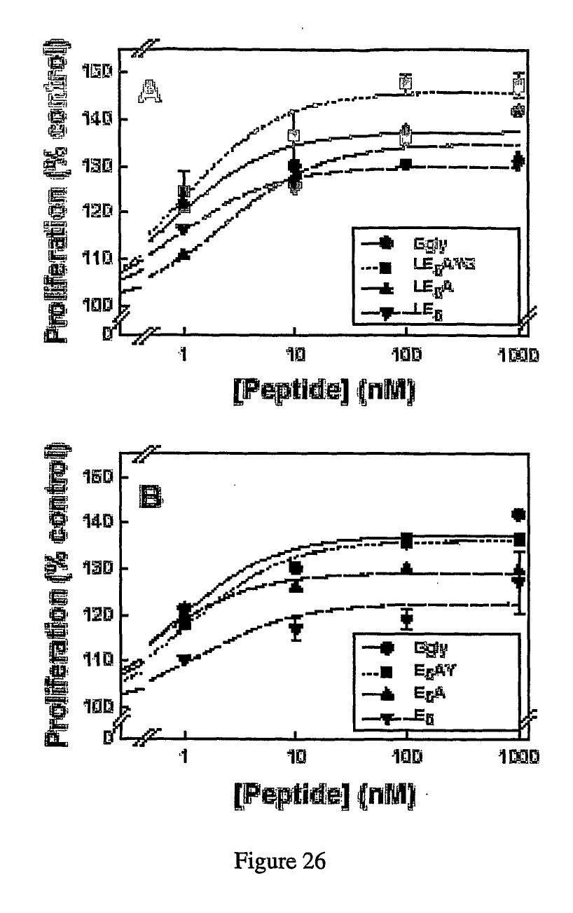

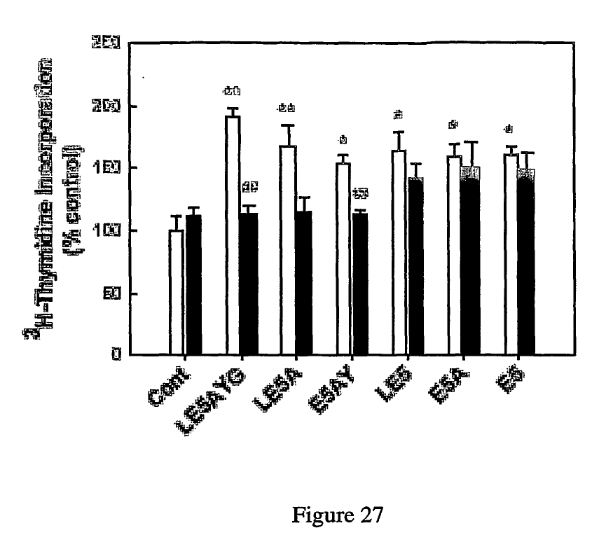

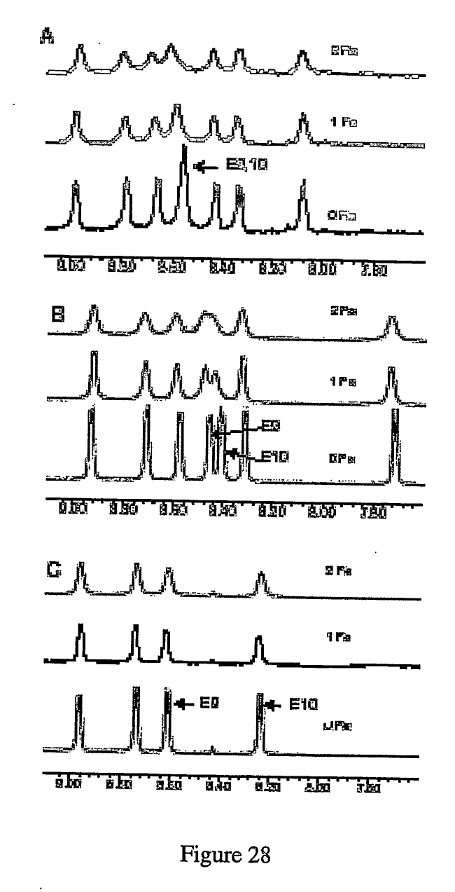

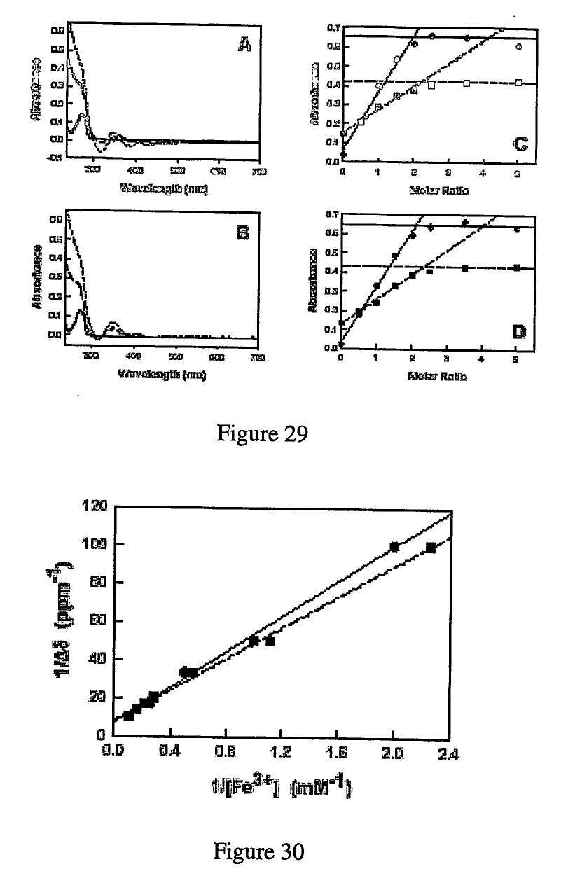

We first compared the activity of Ggly and the nonapeptide LESAYG in cell proliferation and migration assays. The nonapeptide significantly stimulated proliferation of the non-transformed gastric cell line IMGE, as assessed by incorporation of [³H]-thymidine, and the stimulation was completely blocked by inclusion of the chelating agent DFO, as shown in FIG. 25A. In wound healing assays, the nonapeptide appeared to be more effective than Ggly in stimulating cell migration as shown in FIG. 25B. Again the stimulation of migration by the nonapeptide LE₅AYG was reversed by inclusion of DFO in the medium.

We then investigated the activity of shorter fragments of Ggly in cell proliferation assays. All fragments stimulated proliferation of IMGE cells in a dose-dependent manner as illustrated in FIG. 26, but curve fitting of the experimental data indicated that the maximum stimulation achieved at saturating concentrations of the hexapeptides LE₅ and E₅A and the pentapeptide E₅ was significantly less than that achieved with the nonapeptide LE₅AYG, as summarised in Table 10. In contrast, no significant difference was observed between the maximum stimulation achieved at saturating concentrations of the heptapeptides LE₅A and E₅AY and the nonapeptide LE₅AYG. The fact that there was no significant difference between the ED₅₀ values observed for any of the peptides (Table 10) suggested that all peptides bound to the Ggly receptor with similar affinities to the nonapeptide LE₅AYG.

TABLE 10
Affinity and Potency of Ggly and Ggly Fragments as
Stimulants of Cell Proliferation.

| | ED₅₀ | SEM | | S | | | |
|---|---|---|---|---|---|---|---|
| Peptide | (nM) | (nM) | No | (%) | SEM | No | Significance |
| Ggly | 0.8 | 0.4 | 5 | 37.3 | 2.4 | 5 | NS |
| LE₅AYG | 1.0 | 0.4 | 5 | 45.8 | 2.5 | 5 | — |
| LE₅A | 2.4 | 1.1 | 5 | 34.7 | 2.6 | 5 | NS |
| LE₅ | 0.9 | 0.7 | 5 | 29.9 | 3.5 | 5 | 0.003 |
| E₅AY | 1.1 | 0.7 | 5 | 36.1 | 3.4 | 5 | NS |
| E₅A | 0.5 | 0.4 | 5 | 29.1 | 3.1 | 5 | 0.002 |
| E₅ | 1.4 | 1.0 | 5 | 22.5 | 2.5 | 5 | <0.001 |

The effects of increasing concentrations of Ggly or of Ggly fragments on proliferation of IMGE cells were measured in thymidine uptake assays as described in the Materials and Methods section. The data from the five independent experiments shown in FIG. 26 were fitted to the equation
S=100+S C/(ED₅₀+C)
with the program Sigmaplot as described herein, to obtain the indicated values and SEM for ED₅₀ and the maximum % stimulation S indicated above. The significance of differences in S values from the value for the nonapeptide LE₅AYG were assessed by one way analysis of variance, followed by Bonferroni's t-test. There was no significant difference in ED₅₀ values.

### EXAMPLE 20

### Binding of Ferric Ions to GGLY Fragments

In order to determine whether or not the biological activity of Ggly fragments required ferric ions, we next investigated the effect of DFO on cell proliferation induced by the Ggly fragments. The activities of the nonapeptide LE₅AYG and the heptapeptide E₅AY were significantly blocked by DFO in [³H]-thymidine incorporation assays. The activity of the heptapeptide LEsA was lower in the presence of DFO, although the reduction did not reach significance (p=0.059). However, no significant reduction of the activity of the hexapeptides LE₅ or E₅A, or the pentapeptide E₅, was observed in the presence of DFO. These results are illustrated in FIG. 27.

In order to define the ligands involved in ferric ion binding, we next investigated the effect of ferric ions on the NMR spectra of the peptides. Ferric ion titration experiments were carried out by addition of 20, 50, or 200 mM ferric citrate solution to the peptides. The pH was maintained at 5.3 by addition of small amounts of NaO²H or ²HCl. Concentration, pH, temperature and use of ferrric citrate were in keeping with the conditions that were employed in earlier examples in order to prevent precipitation of peptide or ferric hydroxide. Since these fragments did not include a tryptophan residue, fluorescence quenching could not be used. However, the apparent dissociation constants, Kd, for the complexes between ferric ions and the nonapeptide LE₅AYG or the octapeptide LE₅AY were obtained from the change in chemical shift of the GlulO amide proton on addition of ferric ions, as described above.

¹H chemical shifts and NH-C^{α}H coupling constants were determined from 1D, TOCSY and NOESY spectra of the nonapeptide LE₅AYG in 95% H₂O/5% ²H₂O, pH 5.3, 278 K. In contrast to the well-defined structure observed for Ggly in solution, the nonapeptide LE₅AYG appeared to be unstructured. Thus NOEs were only observed between resonances on the same or neighbouring residues, NH and C^{α}H chemical shifts (with the exception of the C-terminal NH resonance) did not differ from random coil values by more than 0.3 ppm, and NH-C^{α}H coupling constants ranged from 6-8 Hz. These features are consistent with conformational averaging of the peptide backbone. The loss of structure on deletion of residues 14-18 of Ggly is in agreement with our suggestion in Example 1 that the disc-like structure of Ggly is stabilized by hydrophobic interactions involving Trp14 and Phe17.

On addition of paramagnetic ferric ions as ferric citrate (1:1), changes were observed in the intensities, resolution and chemical shifts of the NH resonances of the nonapeptide LE₅AYG. As shown in FIG. 28, the largest effects were observed for the overlapping NH peaks of the Glu9 and Glu10 residues (numbering as for the parent Ggly). The maximum change in chemical shift observed was 0.06 ppm at 2 mole of added Fe³⁺/mole peptide for Glu10. At 5 mole Fe³⁺/mole there was a general degradation in the spectral quality due to the high concentration of paramagnetic ions in solution, but the peaks had returned to within 0.01 ppm of their original chemical shift values.

We then investigated the effect of added ferric ions on the NMR spectra of shorter Ggly fragments. For the octapeptide LE₅AY, addition of ferric ions as ferric citrate again resulted in a downfield shift in the NH resonances from Glu9 and GlulO, with a maximum change in chemical shift of 0.05 ppm at 2 mole of added Fe³⁺/mole peptide. For the heptapeptides LE₅A and E₅AY smaller downfield shifts were observed in the NH resonances from Glu9 and Glu10, with a maximum change in chemical shift of 0.01 ppm at 2 mole of added Fe³⁺/mole peptide. For the pentapeptide EsA no shift was observed in the NH resonances from either Glu9 or Glu10.

Thus we have obtained convinceing evidence that the nonapeptide and the octapeptide LE₅AY bind ferric ions via Glu9 and Glu10, and the data with the heptapeptides is consistent with the same conclusion, although the binding in this case is weaker. In fact the decrease in the magnitude of the maximum change in chemical shift from the nonapeptide to the pentapeptide supports the generalization that the affinity of the peptides for ferric ions decreases with decreasing chain length.

The stoichiometry of ferric ion binding to Ggly fragments containing tyrosine residues was measured by absorbance spectroscopy as described in the Materials and Methods section. The ultraviolet absorbance of both the nonapeptide LE₅AYG (FIG. 29A) and the heptapeptide E₅AY (FIG. 29B) increased markedly on addition of ferric ions. Titration experiments at either the absorbance minimum at 247 nm or the absorbance maximum at 275 nm revealed that for both the nonapeptide LE₅AYG (FIG. 29C) and the heptapeptide E₅AY (FIG. 29D) the stoichiometry of binding was approximately 2 moles ferric ion per mole of peptide, as summarised in Table 11. Similar results were obtained with the octapeptide LE₅AY.

The stoichiometry of ferric ion binding to Ggly fragments containing tyrosine residues and the maximum increase in absorbance on addition of ferric ions were determined at the absorption maximum of 275 nm as described for FIG. 29. Values from the indicated number of independent experiments were combined to obtain the mean values (±S.E.M.) presented below.

TABLE 11
Stoichiometry of Ferric Ion Binding by Ggly Fragments.

| | | | Fold | | |
|---|---|---|---|---|---|
| Peptide | Stiochiom. | SEM | Increase | SEM | Replicates |
| Ggly | 2.0* | 0.3* | 2.1* | 0.3* | 3* |
| LE₅AYG | 1.93 | 0.15 | 2.0 | 0.4 | 3 |
| LE₅AY | 2.15 | 0.03 | 2.9 | 0.1 | 3 |
| E₅AY | 2.58 | 0.03 | 3.5 | 0.1 | 3 |

*Taken from Baldwin et al., 2001.

The affinity of Ggly for ferric ions was originally measured by quenching of the fluorescence of the two tryptophan residues (Example 2). The binding data were well fitted by a model with two equivalent but independent binding sites. The same approach could not be used in this experiment because the Ggly fragments lack tryptophan. However, the affinity of the Ggly fragments for ferric ions could be measured from the shift in the GlulO amide resonance on addition of ferric ions, and the results are shown in FIG. 30. The apparent K_{d} values of 7.0 mM obtained for the nonapeptide LE₅AYG and 5.4 mM for the octapeptide LESAY are considerably weaker than the K_{d} value of 0.6 μM obtained previously for Ggly in fluorescence experiments. Furthermore, the reduction observed from the nonapeptide to the pentapeptide in the magnitude of the maximum change in chemical shift on addition of ferric ions is consistent with the suggestion that the affinity of peptides shorter than the nonapeptide for ferric ions decreases with decreasing chain length. However, the reduction in chemical shift prevented accurate estimation of K_{d} values for shorter Ggly fragments.

### EXAMPLE 21

### Proliferative Effect in the Short Bowel Syndrome of GGLY Fragments that Bind Ferric Ions

To test whether or not fragments of Ggly which are bioactive, by virtue of their ferric ion binding, are useful for the treatment of short bowel syndrome, the effect of the nonapeptide LE₅AYG on ileal mucosal proliferation is observed. FIG. 25 shows that the nonapeptide LE₅AYG is active, and that ferric ions are essential for its activity.

The small bowel has a remarkable ability to adapt and regenerate after injury, inflammation and resection. Endogenous growth factors and peptide hormones are thought to be involved in this adaptation. Administration of exogenous growth factors and peptide hormones is a potential therapeutic approach for the treatment of short bowel syndromes.

Rats with a massive small bowel resection (MSBR) are used. 80% of the small bowel is removed, leaving 5 cm of jejunum distal to the ligament of Treitz and 5 cm of ileum proximal to the ileocecal valve. The two ends of the remaining bowel are then anastomosed. The groups (n=10) of animals are as follows:
1. Control rats with transections only
2. Control rats with transections only infused with LE₅AYG
3. Rats with MSBR infused with saline
4. Rats with MSBR infused with LE₅AYG

The LE5AYG peptide is infused intraperitoneally via Alzet osmotic minipumps at a rate of 2.5 nmol/kg/hr. At the end of 21 days food intake, weight gain, jejunal and ileal diameters, total and mucosal wet weights per centimetre, crypt depths and villus heights, mucosal sucrase activity, milligrams of protein per centimetre, micrograms of DNA per centimetre and D-xylose absorption are measured. We expect that the infusion of the LE₅AYG peptide will accelerate the adaptive response of the remaining small bowel following massive small bowel resection.

### EXAMPLE 22

### Addition of GGLY Fragments which Bind Ferric Ions to Total Parenteral Nutrition Solution to Maintain Intestinal Function

Total parenteral nutrition is necessary in a number of conditions, including cancer, inflammation of the bowel, trauma, burns and surgery, but treatment goals should focus on early transition to enteral nutrition followed by oral feeds. Growth factors able to stimulate intestinal absorption and adaptation may facilitate this transition. Malnutrition or prolonged periods without enteral nutrition may lead to decreased gut surface area, mass, and function. The addition of growth factors or hormones or fragments thereof, such as the peptide LE₅AYG, may enhance intestinal compensation and intestinal adaptation.

Rats implanted with a central venous catheter (CVC) are used. The groups (n=10) of animals are as follows:
1. Control rats with CVC infused with Total Parenteral Nutrition solution
2. Rats with CVC infused with Total Parenteral Nutrition solution plus the peptide LE₅AYG added to solution to give a rate of 2.5 nmol/kg/hr.
   At the end of 21 days weight gain, jejunal and ileal diameters, total and mucosal wet weights per centimetre, crypt depths, villus heights, mucosal sucrase activity, milligrams of protein per centimetre, and micrograms of DNA per centimetre and D-xylose absorption are measured. We expect that the infusion of the peptide LE₅AYG will reduce the decline in function of the small bowel associated with total parenteral nutrition.
   Discussion

The results presented herein demonstrate the crucial importance of the binding of ferric ions to the -(Glu)₅- sequence, and in particular to Glu7, for the biological activity of Ggly. In contrast, binding of ferric ions is not essential for the biological activity of Gamide.

The NMR studies showed that the 18 residue peptide Ggly has a well-defined structure in aqueous solution. As reported previously for Gamide (Torda et al, 1985), there were two sets of some resonances, due to cis-trans isomerisation around the Gly2 to Pro3 peptide bond. This effect was particularly striking for the protons of Gly2, and for the N-terminal pyroglutamate residue. The ratio of the cis and trans isomers was 3:7. In both isomers the backbone of the peptide forms a loop, which is stabilized by hydrophobic interactions involving Leu 5, Tyr 12, Trp 14 and Phe 17.

The changes observed in the two-dimensional ¹H NMR spectrum of Ggly after addition of 1 or 2 equivalents of ferric ions confirmed that two ferric ions were able to bind specifically to the -(Glu)₅- sequence. The first equivalent of ferric ions broadened the resonances due to Glu7 beyond detection, while the second equivalent broadened the resonances due to Glu8 and Glu9 beyond detection. Binding appeared to be specific, because the peaks from the other residues in the spectrum were unaffected, except for small shifts in those residues close to the -(Glu)₅- sequence in the structure.

The conclusions from the NMR experiments were in agreement with our previous study of the quenching of tryptophan fluorescence of Ggly fragments by ferric ions (Baldwin et al, 2001), in which the loss of metal ion binding in peptides lacking the -(Glu)₅- sequence indicated that one or more of the glutamate residues was essential for metal ion binding. Furthermore, our earlier findings suggested that the Ggly binding sites were specific for ferric ions, since Co²⁺, Cr³⁺, Cu²⁺ and Mn²⁺ ions did not quench Ggly fluorescence (Baldwin et al, 2001), and since addition of 20 equivalents of Al³⁺ ions did not cause a significant shift in any NMR signals. These observations were also consistent with our previous inability to detect high affinity binding of Ca²⁺ or Co²⁺ ions to Gamide by NMR spectroscopy (Torda et al, 1985).

In the studies reported herein, the identification by NMR spectroscopy of Glu7 as a first site ferric ion ligand, and Glu8 and Glu9 as ligands in the second ferric ion binding site, was confirmed by investigation of fluorescence quenching when ferric ions were added to mutant Ggly peptides in which some or all of the glutamates had been replaced by alanines. Replacement of Glu7 with Ala reduced the stoichiometry of ferric ion binding from the value of 2 observed with Ggly to 1, without changing the apparent binding affinity. In contrast, replacement of Glu6 with Ala had little if any effect on the stoichiometry or affinity of binding. The importance of glutamates 8 and 9 in binding the second ferric ion was confirmed by the similar reduction in stoichiometry observed with a peptide in which glutamates 8, 9 and 10 had all been replaced by alanines. Sequence comparisons of gastrins across eight mammalian species are also consistent with an important role for Glu7, 8 and 9, since these three residues are strictly conserved, with the exception of equine gastrin, which has Lys instead of Glu7 (Moore et al, 1997). In contrast, Glu10 is unlikely to play a functional role, since in four species this residue is replaced by Ala (Moore et al, 1997).

The surprising observation that the peptide in which all five glutamates had been replaced by alanine (GglyE6-10A) still bound one ferric ion, albeit with a significantly lower apparent affinity, suggests that the carbonyl oxygens or amide nitrogens of the peptide backbone, rather than the sidechain carboxylates, may contribute to the second ferric ion binding site. A similar suggestion has been made in relation to Gamide, on the basis of the observation that a modified norleucine¹⁵-Gamide5-17, in which the carboxyl groups of the five consecutive glutamates (Glu6-10) and of Asp16 had been protected by t-butyl groups, still retained the ability to bind one calcium ion in trifluoroethanol (Palumbo et al, 1980). The other ferric ion ligands may include water molecules, and citrate in the case of the NMR experiments, in which iron was added as ferric citrate.

There is now general agreement that both Ggly and Gamide are able to stimulate independently, via different receptors, the proliferation of several different cell lines of gastrointestinal origin (Seva et al, 1994; Hollande et al, 1997; Baldwin et al, 2001). Our results confirmed our previous findings that both Ggly and Gamide were able to induce a 2-fold increase in proliferation of the gastric epithelial cell line IMGE-5 (Hollande et al 2001). In contrast, the previously reported effects of gastrins on cell migration appear to be dependent on the C-terminus of the peptide and on the cell type chosen. Thus Gamide treatment inhibits motility in human glioblastoma cell lines (De Hauwer et al, 1998), but Gamide stimulates basement membrane invasion by a gastric cancer cell line (Wroblewski et al, 2002). In the case of Ggly, increased migration of both gastric and colon cancer cell lines has been observed after treatment with peptide (Hollande et al, 2001; Kermorgant et al, 2001). We have confirmed that Ggly treatment considerably reduced wound size in the gastric epithelial cell line IMGE-5 (FIG. 7), but that Gamide was inactive in this assay.

Our results from comparison of the effects of Ggly mutants in cell proliferation and migration assays consistently highlighted the importance of Glu7 in the biological activity of Ggly. At least two models are consistent with the observation that Glu7 is essential for biological activity. The first model is also based on the NMR and fluorescence data presented in FIGS. 5 and 6 respectively, which indicate that for Ggly Glu7 is critical for binding the first ferric ion. The model postulates that the Ggly receptor will not bind Ggly in the absence of ferric ions, and that binding of the first ferric ion to Glu7 is necessary for receptor binding to occur. In contrast, the second model postulates that Glu7 interacts directly with the Ggly receptor, with no intervening ferric ion. The complete inhibition by the iron chelator DFO of Ggly-stimulated cell proliferation and cell migration in vitro, and the significant inhibition of Ggly-stimulated proliferation in rectal mucosa in the defunctioned rat model in vivo (FIG. 19), provide strong support for the first model. Taken together, our data are consistent with the hypothesis that ferric ion binding to Glu 7 is essential for the biological activity of Ggly. Presumably the activity of Ggly in the absence of added ferric ions is dependent on the adventitious binding of ferric ions (248 nM in Dulbecco's modified Eagle's medium) from the medium.

Although there have been many reports describing the binding of metal ions by hormones, a functional role has seldom been demonstrated, and to our knowledge this is the first report of an essential role of a metal ion in the action of a hormone.

Our studies with Ggly fragments have revealed that either the N-terminal or the C-terminal could be deleted without complete loss of biological activity (FIG. 7). Thus the peptide LE₅AYGWMDFG formed by removal of the four N-terminal residues of Ggly was fully active in cell proliferation or wound healing assays, while the activity of the peptide ZGPWLE₅A formed by removal of the seven C-terminal residues was reduced by approximately 50%. Similarly, both Gglyl-13 (ZGPWLE₅AYG) and Ggly6-18 (E₅AYGWMDFG) were biologically active in stimulating gene expression in canine gastric parietal cells (Kaise et al., 1995). In addition, simultaneous deletion of both four N-terminal and five C-terminal residues to form the nonapeptide LE₅AYG has no effect on maximal activity in either proliferation or migration assays (FIG. 25), or on potency in proliferation assays (Table 10).

These observations are in sharp contrast to the structure-function profile of amidated gastrins, in which only the four C-terminal residues are essential for activity (Tracy and Gregory, 1964) and for binding to the cloned CCK-B receptor (Kopin et al, 1992; Ito et al, 1993). Our results strongly support the contention, originally based on the failure of CCK-2 receptor antagonists to block Ggly activity (Seva et al, 1994; Hollande et al, 1997), that the recognition sequences for the Ggly receptor and the Gamide receptor are quite different.

The biological activity of the nonapeptide LE₅AYG is still absolutely dependent on the presence of ferric ions. As with Ggly (FIG. 9), activity in both proliferation and migration assays is completely blocked by inclusion of the chelating agent DFO in the medium. Absorbance spectroscopy indicated that the nonapeptide still bound 2 ferric ions (FIG. 29), but NMR experiments revealed that the apparent affinity of ferric ions for the nonapeptide (apparent K_{d}=7.0 mM, pH 5.3) was considerably lower than previously calculated for Ggly from fluorescence experiments (K_{d}=0.6 μM, pH 4.0). The difference in affinity between the nonapeptide and Ggly is confirmed by the different behaviour of the amide proton resonances on addition of ferric ions, with the Glu9 and Glu10 resonances of the nonapeptide shifting downfield (FIG. 28A) and the Glu7, 8 and 9 resonances of Ggly stoichiometrically reduced (FIG. 5). However, the precise values cannot be compared directly because of the indicated difference in pH values between the NMR and fluorescence experiments, and because of the inclusion of citrate in the NMR experiments to increase ferric ion solubility at the higher pH. Unfortunately attempts to repeat the LE₅AYG titration with ferric chloride instead of ferric citrate resulted in the immediate formation of a precipitate.

Part of the reduction in affinity for ferric ions between Ggly and the nonapeptide may be the result of different glutamates acting as ferric ion ligands. Thus, for Ggly the first ferric ion binds to Glu7 and the second to Glu8 and Glu9 (FIG. 5), while for the nonapeptide the observation that binding of ferric ions simultaneously affects Glu9 and Glu10 (numbering as for the parent Ggly) suggests that these two residues act as ligands at both the first and second ion binding sites. The change in iron ligation may in turn result from the loss of the well-defined loop in the Ggly structure consequent on removal of the hydrophobic residues Trp14 and Phe17 (FIG. 4). The greater changes in absorption at the minima at 247 nm observed on addition of ferric ions to the nonapeptide LE₅AYG, the octapeptide LE₅AY, or the heptapeptide E₅AY (FIG. 29) when compared with the corresponding changes in the Ggly spectrum (Baldwin et al., 2001) may be a consequence of the involvement of different glutamates in iron binding.

Ggly fragments shorter than the nonapeptide are also biologically active, but the dependence on ferric ions varies with chain length. The shortest fully active fragments of Ggly are the heptapeptides LE₅A and E₅AY (Table 10), for both of which activity remains iron-dependent (FIG. 27). Further N- or C-terminal truncation results in a progressive reduction in maximal activity, although there is no significant change in potency (Table 9). Interestingly, the hexapeptides LE₅ and E₅A and the pentapeptide E₅ possess significant activity (Table 10), which is not reduced in the presence of the chelating agent DFO (FIG. 27). Furthermore, in the NMR experiments the reduction observed from the nonapeptide to the pentapeptide in the magnitude of the maximum change in chemical shift on addition of ferric ions is consistent with the suggestion that the affinity of the shorter peptides for ferric ions decreases with decreasing chain length. The failure of DFO to inhibit the activity of the Ggly fragments LE₅, EsA, or E₅ (FIG. 27) is presumably therefore a reflection of the reduced affinity of the shorter peptides for ferric ions.

In the case of Gamide, replacement of Glu7 with Ala reduced the stoichiometry of ferric ion binding from 1.6 to 0.9, without changing the apparent affinity (Table 7). The importance of glutamates 8 and 9 in binding the second ferric ion was confirmed by the similar reduction in stoichiometry observed with a peptide in which glutamates 8 and 9 had both been replaced by alanines. Sequence comparisons of gastrins across eight mammalian species are also consistent with an important role for Glu7, 8 and 9, since these three residues are strictly conserved, with the exception of equine gastrin, which has Lys instead of Glu7 (Moore et al. 1997). In contrast, Glu10 is unlikely to play a functional role, since in four species it is replaced by Ala (Mopre et al. 1997).

Mutation of Glu7, or Glu8 and 9, to Ala slightly decreased the affinity of Gamide for the CCK-2 receptor (Table 8). The ratio IC₅₀ GamideE7A/IC₅₀ Gamide was 1.4 for transfected COS-7 cells and 2.4 for T-lymphoblastoid Jurkat cells, but the difference was significant only in the latter case. Similarly the ratio IC₅₀ GamideE8,9A/ IC₅₀ Gamide was 4.1 for transfected COS-7 cells and 2.2 for T-lymphoblastoid Jurkat cells, and the difference was significant only in the latter case. We conclude that Glu7, 8 and 9 are not essential for binding of Gamide to the CCK-2 receptor. Furthermore, since Glu7, 8 and 9 have been defined as the ligands for ferric ion binding to Gamide, we conclude that binding of ferric ions is not essential for recognition of Gamide by the CCK-2 receptor. The 10-40 fold difference in the affinities measured in COS-7 cells and Jurkat cells may perhaps be explained by the greater amounts of CCK-2 receptor expressed in COS-7 cells. For example, there may be insufficient amounts of G protein, or other intracellular protein coupled to the CCK-2 receptor, to convert all the CCK-2 receptor to the higher affinity form.

Comparison of the ability of Gamide mutants, in which one or more of the glutamates were replaced by alanine, to stimulate proliferation or inositol phosphate production revealed that Glu7, 8 and 9 were not essential for biological activity. In contrast, comparison of Ggly mutants, in which one or more of the glutamates were replaced by alanine, demonstrated that Glu7 was essential for proliferation (FIG. 8). Our results are consistent with previous data on the physiological properties of a series of synthetic peptides structurally related to Gamide (Tracy & Gregory 1964), which indicated that only the C-terminal tetrapeptide Trp-Met-Asp-Phe-NH₂ was required for a range of physiological effects. These results have been confirmed in binding experiments with Gamide fragments and the cloned CCK-2 receptor (Kopin et al. 1992, Ito et al. 1993). However, residues N-terminal to the tetrapeptide must contribute to the binding of G17 to the CCK-2 receptor, since both potency in bioassays and affinity for the CCK-2 receptor increased with chain length. The observation that pentagastrin (tert-butoxycarbonyl-p-ala-trp-met-asp-phe-amide) is as potent as Gamide in vivo (Morley et. al. 1965) suggests that the additional tert-butoxycarbonyl-β-ala group of pentagastrin makes a contribution to the binding energy approximately equal to the contribution of the polyglutamate ferric ion binding region. Both contributions could arise from independent binding to different regions of the receptor. In the case of pentagastrin that contribution would be independent of ferric ions, but in the case of Gamide it could be either iron-dependent or -independent. The possible contribution of ferric ions had not been investigated prior to our work.

The results of our experiments with the iron chelator DFO are consistent with the bioactivity and binding data for Gamide derivatives described in the previous paragraph. Thus DFO had no effect on the affinity of Gamide for the CCK-2 receptor expressed on COS-7 cells (FIG. 24A) or on Jurkat cells (FIG. 24B), or on the ability of Gamide to stimulate inositol phosphate production in COS-7 cells (FIG. 24C). Our data with DFO confirm that the binding of ferric ions to Glu7-9 of Gamide is not essential for CCK-2 receptor binding or biological activity.

This conclusion at first sight appears to conflict with the observation that DFO reduced Gamide-stimulated proliferation of the gastric cell line IMGE by 60% (FIG. 9A). One possible explanation for this apparent discrepancy is that the proliferative response to Gamide was measured over a 72 hour period, whereas the inositol phosphate assay used to assess the effect of DFO on Gamide stimulation was for 1 hour only. The inhibition of cellular ferric ion uptake by DFO is time-dependent, with little or no inhibition after 2 hours, and significant inhibition after 24 hours (Kicic et al. 2001). We therefore postulate that proliferation involves a rate-limiting ferric ion-dependent step, such as cellular ferric ion uptake, which is not controlled by the CCK-2 receptor, and that this step is inhibited only after prolonged incubation with DFO.

Thus the involvement of the -(Glu)₅- sequence in Ggly activity is in direct contrast to amidated gastrins, the glutamates of which are not necessary for full activity. The observation that DFO completely blocked the stimulation of cell proliferation and migration by Ggly is consistent with the hypothesis that ferric ion binding is essential for the biological activity of non-amidated gastrins. Recognition of the essential role of ferric ions may assist in the identification of the Ggly receptor, and also facilitates the development of Ggly antagonists for blockade of the proliferative effects of Ggly in the normal gastrointestinal tract and in colorectal cancer.

Although bismuth has been used as a gastrointestinal therapeutic for over two centuries, there is still no established consensus on its mechanism of action. On the basis of our findings regarding the role of ferric ions, we hypothesised

(a) that trivalent bismuth ions might compete with ferric ions for the Ggly binding sites, and hence

(b) that bismuth ions might block the biological activity of Ggly.

The fluorescence and NMR spectroscopic data presented herein are consistent with the first hypothesis. Fluorescence quenching experiments indicate that Ggly binds two bismuth ions at pH 4.0, with an affinity (5.8 μM) 10-fold lower than for ferric ions. The affinity of Ggly for bismuth or ferric ions at pH 7.6 cannot be calculated exactly, for reasons discussed previously (Baldwin et al, 2001). However, the apparent first dissociation constants for the complex between ferric ions and nitrilotriacetic acid are 830 and 0.19 nM at pH 4.0 and 7.6, respectively. Based on the same ratio, the affinity of Ggly for bismuth ions would be approximately 1.3 nM at pH 7.6. At a Ggly concentration of 10 nM, the occupancy of the bismuth binding sites would therefore be greater than 98%.

The changes observed in the NMR spectrum of Ggly on addition of bismuth ions reveal that glutamates 7, 8 and 9 act as bismuth ion ligands. These observations are similar to the effects of ferric ions on the NMR spectrum of Ggly. Because addition of one molar equivalent of ferric ions broadened the resonances of glutamate 7, without significantly affecting other resonances, we concluded that glutamate 7 acts as a ligand for the first ferric ion. Similarly, because addition of a second molar equivalent of ferric ions broadened the resonances of glutamates 8 and 9, without significantly affecting other resonances, we concluded that glutamates 8 and 9 act as ligands for the second ferric ion. Thus the Ggly side chains acting as bismuth ion ligands are similar to the side chains acting as ferric ion ligands, despite the greater ionic radius of the Bi³⁺ ion (0.096 nm) compared to the Fe³⁺ ion (0.064 nm). Our results are consistent with the suggestion that bismuth and ferric ions compete for the same metal ion binding site on Ggly.

The biological data presented in this specification are consistent with the second hypothesis, that bismuth ions selectively block the biological activity of Ggly. Thus the addition of bismuth ions significantly inhibited both Ggly-stimulated inositol phosphate production (FIG. 13) and proliferation (FIG. 14) in the human colorectal carcinoma cell line HT29, and migration of the gastric epithelial cell line IMGE5 (FIG. 15). Similar inhibitory effects of bismuth ions on Ggly-stimulated inositol phosphate production were also observed in IMGE-5 cells, but in this case the magnitude of Ggly stimulation was only 110% compared to the control. In addition bismuth ions completely block the stimulatory effect of Ggly on rectal mucosa in the defunctioned rat model (FIG. 19). The observations that bismuth ions also bind to Glu7 but inhibit Ggly-stimulated biological activity are consistent with the conclusion that bismuth ions compete for the ferric ion binding site, but that the complex formed is inactive.

In contrast, bismuth ions do not affect the biological activity of Gamide. Thus the addition of bismuth ions did not significantly inhibit either Gamide-stimulated inositol phosphate production in COS-7 cells transiently transfected with the CCK-2 receptor (FIG. 13) or proliferation in CHO cells stably transfected with the CCK-2 receptor (FIG. 14). These observations are consistent with our demonstration that binding of ferric ions to Glu7 of Gamide is not required for biological activity, and with the early demonstration by Tracy and Gregory that the C-terminal tetrapeptide amide is the minimum biologically active fragment of gastrin (Tracy and Gregory, 1964).

Bismuth therapy has been used for a variety of gastrointestinal conditions, including gastric and duodenal ulcers, dyspepsia, diarrhoea and colitis (Gorbach, 1990). Not surprisingly, this broad spectrum of effects is associated with a large number of putative mechanisms of action. Bismuth has a direct antibacterial effect on Helicobacter pylori, and preferentially coats the ulcer craters, preventing back-diffusion of H⁺ ions, (Gorbach, 1990; Lambert and Midolo, 1997). Additional effects of bismuth include accelerated repair of the ulcer crater through influx of macrophages, stimulation of prostaglandin synthesis, and diminished pepsin activity (Gorbach, 1990; Lambert and Midolo, 1997). Bismuth itself does not directly inhibit gastric acidity, but on cessation of treatment acid production increases markedly (Wieriks et al, 1982). The increase may be at least partly due to recovery from the decreased antral G cell density observed in rats during bismuth treatment (Waldum et al, 1994). In colitis, the antidiarrhoeal and anti-inflammatory effects of bismuth have proved beneficial (Fine and Lee, 1998).

The relationship of inhibition of Ggly activity by bismuth and the therapeutic effect of bismuth is unclear, in part because the biological roles of Ggly are still the subject of debate. In terms of ulcer disease, inhibition of Ggly may modulate the long-term acid stimulatory effects of an increased serum Gamide level, since Ggly seems to be required for the Gamide-mediated hyperchlorhydria (Chen et al, 2000). To our knowledge, bismuth has not previously been proposed for the treatment of experimental or clinical colon cancer. However, Ggly stimulates proliferation and migration in gastric and colonic cell lines, and accelerates colon carcinogenesis in rats treated with azoxymethane.

Several Fe chelators have been tested as inhibitors of proliferation of hepatoma cell lines in vitro. It was found that the membrane-permeable chelators pyridoxal isonicotinoyl hydrazone, 1,2-dimethyl-3-hydroxypyridin-4-one, and DFO were effective inhibitors. The membrane-impermeable chelator ethylene diamine tetraacetic acid (EDTA) was ineffective, but surprisingly the membrane-impermeable chelator diethylene triamine pentaacetic acid (DTPA) was the best inhibitor of all those tested (Kicic et al, 2001). In nude mice, DFO is an effective inhibitor of human hepatomas (Hann et al, 1992), but not of neuroblastomas (Selig et al, 1998). We are not aware of any reports of effects of these agents on colorectal carcinomas. Hepatomas and neuroblastomas have not been reported to be associated with elevated levels of non-amidated gastrin. As the person skilled in the art will be aware, the response of a given type of cancer cell to a putative therapeutic agent cannot readily be predicted on the basis of the response to that agent of a different type of cancer cell.

Therefore in the light of the results presented in this specification, bismuth and other compounds of this invention are expected to be useful in the treatment of colon cancer, and their efficacy can readily be tested in models of colon carcinogenesis. For example, the effects of bismuth on the development of aberrant crypt foci could be investigated in rats treated with the colon carcinogen azoxymethane, using the method of Aly et al., 2001. In addition to the animal models referred to in the Examples, many other animal models are known, including colon cancer induced in Sprague-Dawley rats by treatment with dimethylhydrazine, the Smad3 mutant mouse, the APC^{min} mouse, and the ApC^{Δ716} mouse.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

This invention was made with support from the Australian Research Council, the National Health and Medical Research Council of Australia, and the US National Institutes of Health (NIGMS grant R01 GM 65926-01). The United States government may have certain rights in the invention in the United States.

References cited herein are listed on the following pages, and are incorporated herein by this reference.

### REFERENCES

Aly, A., Shulkes, A. and Baldwin, G. S. Short term infusion of glycine-extended gastrin₁₇ stimulates proliferation and formation of aberrant crypt foci in rat colonic mucosa. Int. J. Cancer. 94: 307-313 (2001).
Anderson R A and Vallee B L. Selective cobalt oxidation as a means to differentiate metal-binding sites of cobalt alkaline phosphatase. Biochemistry 16: 4388-4393 (1977).
Balakrishnan M S and Villafranca J J. Preparation and characterization of cobalt(III)- and chromium(III)-glutamine synthetase derivatives. Biochemistry 18:1546-1551(1979).
Baldwin, G. S. The role of gastrin and cholecystokinin in normal and neoplastic gastrointestinal growth. J. Gastro. Hepatol. 10, 215-232(1995).
Baldwin, G. S. Comparison of sequences of the 78 kDa gastrin-binding protein and some enzymes involved in fatty acid oxidation. Comp. Biochem. Physiol. 104B:55-61(1993).
Baldwin, G. S., Hollande, F., Yang, Z., Karelina, Y., Paterson, A., Strang, R., Fourmy, D., Neumann, G. and Shulkes, A. Biologically active recombinant human progastrin₆₋₈₀ contains a tightly bound calcium ion. J. Biol. Chem. 276: 7791-7796 (2001).
Baldwin G S, Curtain C C, Sawyer W H. Selective, high-affinity binding of ferric ions by glycine-extended gastrin(17). Biochemistry; 40:10741-10746 (2001).
Baldwin G S and Shulkes A. Gastrin, gastrin receptors and colorectal carcinoma. Gut. 42:581-584(1998).
Barnham, K. J., Torres, A. T., Alewood, D., Alewood; P. F., Domagala, T., Nice, E. C., & Norton, R. S. Protein Sci. 7, 1738-1749(1998).
Barnham K J, Catalfamo F, Pallaghy P K, Howlett G J, Norton R S. Helical structure and self association in a 13 residue neuropeptide Y Y2 receptor agonist: relationship to biological activity. Biochem. Biophys. Acta. 1435:127-137 (1999).
Chen D, Zhao C M, Dockray G J, Varro A, Van Hoek A, Sinclair N F, Wang T C, Koh T J. Glycine-extended gastrin synergizes with gastrin 17 to stimulate acid secretion in gastrin-deficient mice. Gastroenterology. 119:756-65 (2000).
Cobb S, Wood T, Tessarollo L, Velasco M, Given R, Varro A, et al. Deletion of functional gastrin gene markedly increases colon carcinogenesis in response to azoxymethane in mice. Gastroenterology 123:516-530 (2002).
Cui H, Cruz-Correa M, Giardiello F M, Hutcheon D F, Kafonek D R, Brandenburg S, Wu Y, He X, Powe N R, Feinberg A P. Loss of IGF2 Imprinting: A Potential Marker of Colorectal Cancer Risk. Science 299:1753-1755 (2003).
De Hauwer, C., Camby, I., Darro, F., Migeotte, I., Decaestecker, C., Verbeek, C., Danguy, A., Pasteels, J. L., Brotchi, J., Salmon, I., Van Ham, P., and Kiss, R. J. Neurobiol. 37, 373-382 (1998).
Dockray, G J. Gastrin and gastric epithelial physiology J. Physiol. 518:315-324 (1999).
Galleyrand J C, Lima-Leite A C, Lallement J C, Lignon M F, Bernad N, Fulcrand P, Martinez J. Synthesis and characterization of a new labeled gastrin ligand, 125-I-BH-[Leu15]-gastrin-(5-17), on binding to canine fundic mucosal cells and Jurkat cells. Int J Pept Protein Res. 44:348-356 (1994).
Gorbach S L. Bismuth therapy in gastrointestinal diseases. Gastroenterology 99:863-875 (1990).
Hann H W, Stahlhut M W, Rubin R, Maddrey W C. Antitumor effect of deferoxamine on human hepatocellular carcinoma growing in athymic nude mice. Cancer 70:2051-2056 (1992).
Henwood M, Clarke P A, Smith A M, Watson S A. Expression of gastrin in developing gastric adenocarcinoma. Br J Surg 88:564-568 (2001).
Higashide S, Gomez G, Greeley G H Jr, Townsend J C. Glycine-extended gastrin potentiates gastrin-stimulated gastric secretion in rats. Am. J. Physiol. 270(1 Pt1):G220-G224 (1996).
Hirata M, Itoh M, Tsuchida A, Ooishi H, Hanada K, Kajiyama G. Cholecystokinin receptor antagonist, loxiglumide, inhibits invasiveness of human pancreatic cancer cell lines. FEBS Lett 383:241-244 (1996).
Hollande F, Blanc E M, Bali J P, Whitehead R H, Pelegrin A, Baldwin G S, Choquet A. HGF regulates tight junctions in new nontumorigenic gastric epithelial cell line. Am. J. Physiol. 280:G910-G921 (2001).
Hollande F, Choquet A, Blanc E M, Lee D J, Bali J P, Baldwin G S. Involvement of phosphatidylinositol 3-kinase and mitogen-activated protein kinases in glycine-extended gastrin-induced dissociation and migration of gastric epithelial cells. J. Biol. Chem. 276:40402-40410 (2001).
Hollande F, Imdahl A, Mantamadiotis T, Ciccotosto G D, Shulkes A, Baldwin G S. Glycine-extended gastrin acts as an autocrine factor in a nontransformed colon cell line. Gastroenterology. 113:1576-1588 (1997).
Ito, M., Matsui, T., Taniguchi, T., Tsukamoto, T., Murayama, T., Arima, N., Nakata, H., Chiba, T., & Chihara, K. J. Biol. Chem. 268, 18300-18305 (1993).
Iwase K, Evers B M, Hellmich M R, Guo Y S, Higashide S, Kim H J et al. Regulation of growth of human gastric cancer by gastrin and glycine-extended progastrin. Gastroenterology 113:782-790 (1997).
Kaise, M., Muraoka, A., Seva, C., Takeda, H., Dickinson, C. J., & Yamada, T. J. Biol. Chem. 270, 11155-11160 (1995).
Kermorgant S, Lehy T. Glycine-extended gastrin promotes the invasiveness of human colon cancer cells. Biochem. Biophys. Res. Commun. 285:136-141 (2001).
Kicic A, Chua A C, Baker E. Effect of iron chelators on proliferation and iron uptake in hepatoma cells. Cancer 92:3093-3110 (2001).
Kidd M, Modlin I, Tang L. Gastrin and the enterochromaffin like cell: an update. Dig Surg 15:209-217 (1998). Kirton C M, Wang T, Dockray G J. Regulation of parietal cell migration by gastrin in the mouse. Am. J. Physiol. 283:G787-G793 (2002).
Koh T J, Dockray G J, Varro A, Cahill R J, Dangler C A, Fox J G, Wang T C. Overexpression of glycine-extended gastrin in transgenic mice results in increased colonic proliferation. J. Clin. Invest. 103:1119-1126 (1999).
Koh, T. J., Bulitta, C. J., Fleming, J. V., Dockray, G. J., Varro, A. and Wang, T. C. Gastrin is a target of the β-catenin/TCF-4 growth-signaling pathway in a model of intestinal polyposis. J. Clin. Invest. 106: 533-539 (2000).
Kopin, A. S., Lee, Y. M., Mc Bride, E. W., Miller, L. J., Kolakowski, L. F., & Beinborn, M. Proc. Natl. Acad. Sci. U. S. A. 89, 3605-3610 (1992).
Koradi, R., Billeter, M., & Wutrich, R. J. Mol. Graph. 14, 51-55 (1996).
Lehy T. Trophic effect of some regulatory peptides on gastric exocrine and endocrine cell of the rat. Scand J Gastroenterol 19(Suppl 101):27-30 (1984).
Linse S, Johansson C, Brodin P, Grundstrom T, Drakenberg T, Forsen S. Electrostatic contributions to the binding of Ca²⁺ in calbindin D9k. Biochemistry. 30:154-162 (1991).
Litvak D A, Hellmich M R, Iwase K, Evers B M, Martinez J, Amblard M et al. JMV1155: a novel inhibitor of glycine-extended progastrin-mediated growth of a human colon cancer in vivo. Anticancer Res 19:45-9 (1999).
Malby S, Pickering R, Saha S, Smallridge R, Linse S, Downing A K. The first epidermal growth factor-like domain of the low-density lipoprotein receptor contains a noncanonical calcium binding site. Biochemistry 40:2555-2563 (2001).
Marshall B J, Armstrong J A, Francis G J, Mokes N T, Wee S H. Ntibacterial action of bismuth in relation to Campylobacter pyloridis colonization and gastritis. Digestion. 37 (Suppl 2):16-30 (1987).
McLellan, E. A. & Bird, R. P. Specificity study to evaluate induction of aberrant crypts in murine colons. Cancer Res. 48: 6183-6186 (1988).
Moore, C., Jie, R., Shulkes, A., & Baldwin, G. S. DNA Sequence. 8, 39-44 (1997).
Morley J S, Tracy H J & Gregory R A. Structure-function relationships in the active C-terminal tetrapeptide sequence of gastrin. Nature 207:1356-1359 (1965).
Moser, A. R., Pitot, H. C. & Dove, W. F. A dominant mutation that predisposes to multiple intestinal neoplasia in the mouse. Science. 247: 322-324 (1990).
Okada N, Kubota A, Imamura T, Suwa H, Kawaguchi Y, Ohshio G et al. Evaluation of cholecystokinin, gastrin, CCK-1 receptor, and CCK-2/gastrin receptor gene expressions in gastric cancer. Cancer Lett 106:257-262 (1996).
Palumbo, M., Jaeger, E., Knof, S., Peggion, E., & Wunsch E. FEBS Lett. 119, 158-161 (1980).
Qian J M, Rowley W H, Jensen R T. Gastrin and CCK activate phospholipase C and stimulate pepsinogen release by interacting with two distinct receptors. Am. J. Physiol. 264:G718-G727 (1993).
Reubi J C, Waser B, Schmassmann A, Laissue J A. Receptor autoradiographic evaluation of cholecystokinin, neurotensin, somatostatin and vasoactive intestinal peptide receptors in gastro-intestinal adenocarcinoma samples: where are they really located? Int J Cancer 81:376-386 (1999).
Rooman I, Lardon J, Flamez D, Schuit F, Bouwens L. Mitogenic effect of gastrin and expression of gastrin receptors in duct-like cells of rat pancreas. Gastroenterology 121:940-949 (2001).
Seet L, Fabri L, Nice E C, Baldwin G S. Comparison of iodinated [Nle15]- and [Met15]-gastrin17 prepared by reversed-phase HPLC. Biomed. Chroatogr. 2:159-163 (1987).
Seimann. In: Rodent Tumor Models in Experimental Cancer Therapy Ed. Kallman. pp. 12-15. (Pergamon Press, N.Y.) (1987)
Selig R A, White L, Gramacho C, Sterling-Levis K, Fraser I W, Naidoo D. Failure of iron chelators to reduce tumor growth in human neuroblast xenografts. Cancer Res. 58:473-8 (1998).
Seva C, Dickinson C J, Yamada T. Growth-promoting effects of glycine-extended progastrin. Science 265:410-412 (1994).
Singh P, Velasco M, Given R, Wargovich M, Varro A, Wang T C. Mice overexpressing progastrin are predisposed for developing aberrant colonic crypt foci in response to AOM. Am. J. Physiol. 278:G390-G399 (2000a).
Singh P, Velasco M, Given R, Varro A, Wang T C. Progastrin expression predisposes mice to colon carcinomas and adenomas in response to a chemical carcinogen. Gastroenterology 119:162-171 (2000b).
Stepan V M, Sawada M, Todisco A, Dickinson C J. Glycine-extended gastrin exerts growth-promoting effects on human colon cancer cells. Mol Med. 5:147-59 (1999).
Torda, A. E., Baldwin, G. S., & Norton, R. S. Biochem. 24, 1720-1727 (1985).
Tracy H J and Gregory R A. Physiological properties of a series of synthetic peptides structurally related to gastrin I. Nature 204:935 (1964).
Van Oijen A H A M, Verbeek A L, Jansen J B M J, De Boer W A. Treatment of Helicobacter pylori infection with ranitidine bismuth citrate- or proton pump inhibitor-based triple therapies. Aliment Pharmacol Ther 14:991-999 (2000).
Waldum H L, Qvigstad G, Marvik R, Brenna E, Syversen U, Sandvik A K The effect of tripotassium dicitrato bismuthate on the rat stomach. Aliment Pharmacol Ther 8:425-431 (1994).
Wang T C, Koh T J, Varro A, Cahill R J, Dangler C A, Fox J G, Dockray G J. Processing and proliferative effects of human progastrin in transgenic mice. J Clin Invest. 98:1918-1929 (1996).
Weinstock J and Baldwin G S. Binding of gastrin₁₇ to human gastric carcinoma cell lines. Cancer Res. 48:932-937 (1988).
Wieriks J, Hespe W, Jaitly K D, Koekkoek P H, Lavy U Pharmacological properties of colloidal bismuth subcitrate. Scand J Gastroenterol 17 (Suppl 80):11-16 (1982).
Winzor D J and Sawyer W H. Quantitative Characterisation of Ligand Binding, pp. 28-41, Wiley-Liss, New York (1995).
Wroblewski, L. E., Pritchard, D. M., Carter, S., and Varro, A. Biochem. J. 365: 873-879 (2002).
Yang C H, Ford J, Karelina Y, Shulkes A, Xiao S D, Baldwin G S. Identification of a 70-kDa gastrin-binding protein on DLD-1 human colorectal carcinoma cells. Int. J. Biochem. Cell Biol. 33:1071-1079 (2001).

1-40. (canceled)

41. A method of treatment or prophylaxis of a condition associated with elevated levels of non-amidated gastrin, comprising the step of administering to a mammal in need of such treatment an effective amount of a compound which has the ability to inhibit the binding of ferric ions to any one or more of glycine-extended gastrin₁₇ or progastrin or progastrin-derived peptides, but which does not inhibit the activity of amidated gastrin, thereby to inhibit the activity of non-amidated gastrins.

42. A method according to claim 41, in which the compound inhibits the binding of ferric ions to glutamate 7 of glycine-extended gastrin₁₇.

43. A method according to claim 42, in which the binding of ferric ions to glutamate 8 and glutamate 9 of glycine-extended gastrin₁₇ is also inhibited.

44. A method according to claim 41, in which the compound is a metal ion, or a pharmaceutically-acceptable salt or complex thereof, which is able to occupy the ferric ion binding site of non-amidated gastrins, and thereby to block their biological activity.

45. A method according to claim 44, in which the metal ion is any metal ion capable of occupying the ferric ion binding site of non-amidated gastrins, with the provisos that
(i) when the condition is one caused by Helicobacter pylori infection, the metal ion is not bismuth, and
(ii) when the condition is cancer, the salt or complex is not BiISrC₆H₅O₆.

46. A method according to claim 45, in which the metal ion is Bi³⁺ or Ga³⁺.

47. A method according to claim 41, in which the compound is an exchange-inert complex between a non-amidated gastrin and either Co (III) or Cr (III) ions.

48. A method according to claim 41, in which the compound is a pharmaceutically-acceptable chelating agent with a high degree of specificity for ferric ions.

49. A method according to claim 48, in which the chelating agent is membrane-impermeable.

50. A method according to claim 49, in which the chelating agent is desferrioxamine (DFO), ethylene diamine tetracetic acid (EDTA) or diethylene triamine pentacetic acid (DTPA).

51. A method according to claim 48, in which the chelating agent is a membrane-permeable chelator.

52. A method according to claim 51, in which the chelating agent is clioquinol.

53. A method according to claim 41, in which the compound does not have a significant inhibitory effect on Gamide-induced inositol phosphate production and/or on cellular proliferation in cells which express the CCK-2 receptor.

54. A method according to claim 46, in which the compound is one or more of colloidal bismuth subcitrate (CBS), bismuth subcitrate, bismuth citrate, bismuth salicylate, bismuth subsalicylate, bismuth subnitrate, bismuth subcarbonate, bismuth tartrate, bismuth subgallate, tripotassium dicitrato bismuthate or bismuth aluminate.

55. A method according to claim 54, in which the compound is one or more of colloidal bismuth subcitrate (CBS), tripotassium dicitrato bismuthate, bismuth subcitrate, or bismuth subsalicylate.

56. A method according to claim 55, in which the compound is CBS or tripotassium dicitrato bismuthate.

57. A method according to claim 56, in which the compound is CBS.

58. A method according to claim 41, in which the condition is selected from the group consisting of gastrin-producing tumours, colorectal carcinomas, gastrinomas, islet cell carcinomas, lung cancer, ovarian cancer, pituitary cancer and pancreatic cancer.

59. A method according to claim 58, in which the condition is colon cancer or pancreatic cancer.

60. A method according to claim 59, in which the condition is colon cancer and the mammal is at elevated risk thereof.

61. A method according to claim 60, in which the mammal is an individual with any one or more of familial adenomatous polyposis, with a family history of colon cancer, and/or with loss of imprinting of IGF-2.

62. A method according to claim 41, in which the condition is selected from the group consisting of atrophic gastritis, G cell hyperplasia, pernicious anaemia, renal failure and ulcerative colitis.

63. A method according to claim 41, in which the condition is selected from the group consisting of gastrointestinal ulcers, gastro-oesophageal reflux, gastric carcinoid, and Zollinger-Ellison syndrome, with the proviso that the metal ion is not bismuth.

64. A peptide which is a fragment of a non-amidated gastrin and which
(a) comprises at least glutamate residue 7 of the -(Glu)₅- sequence of non-amidated gastrin, and
(b) is capable of binding one or more ferric ions, with the proviso that the peptide is not full length Ggly, full length glycine-extended gastrin or full length progastrin, or LE₅AYG.

65. A peptide according to claim 64, consisting of amino acids 5 to 14 of the Ggly sequence.

66. A peptide according to claim 64, selected from the group consisting of Ggly₅₋₁₈, Ggly₁₋₁₁, LE5AY, LESA, LE₅, E₅A, E₅, and E₅AY.

67. A peptide according to claim 64, in which the carboxy terminus of the peptide is amidated.

68. A peptide according to claim 64, in which the amino terminus of the peptide is acetylated.

69. A complex comprising
(a) a non-amidated gastrin, a peptide fragment thereof according to claim 64, or LE₅AYG, and
(b) a trivalent metal ion.

70. A complex according to claim 69, in which the trivalent metal ion is Bi³⁺ or Ga³⁺.

71. A complex according to claim 69, comprising a non-amidated gastrin and bismuth ions.

72. A composition comprising
(a) a peptide according to claim 64, or LE₅AYG, together with a pharmaceutically acceptable carrier, excipient or diluent.

73. A method of promoting intestinal function, comprising the step of administering a peptide according to claim 64 to a subject in need of such treatment.

74. A method according to claim 73, in which the subject is suffering from injury to the bowel, an inflammatory condition of the bowel, or short bowel syndrome, has undergone a partial or complete resection of the bowel, or is undergoing total parenteral nutrition.

75. A method of screening of candidate metal ion-binding compounds for ability to modulate the activity of non-amidated gastrins, comprising the steps of
(a) assessing the ability of the compound to inhibit binding of ferric ions to a non-amidated gastrin and/or
(b) assessing the ability of the compound to modulate proliferation and/or migration of cells of a gastric mucosal cell line in response to a non-amidated gastrin.

76. A method according to claim 75, in which the non-amidated gastrin is Ggly₁₇.

77. A method according to claim 75, in which the gastric mucosal cell line is IMGE-5.

78. A method according to claim 75, in which the compound is additionally assessed for its ability to inhibit Gamide-induced inositol phosphate production, and/or cellular proliferation in cells which express the CCK-2 receptor.

79. A composition comprising a complex according to claim 69, together with a pharmaceutically acceptable carrier, excipient or diluent.

80. A method of promoting intestinal function, comprising the step of administering
(a) A peptide which is a fragment of a non-amidated gastrin and which
(i) comprises at least glutamate residue 7 of the -(Glu)₅- sequence of non amidated gastrin, and
(ii) is capable of binding one or more ferric ions, with the proviso that the peptide is not full length Ggly, full length glycine-extended gastrin or full length progastrin, and
(b) a complex comprising
(i) a non-amidated gastrin, a peptide fragment thereof according to claim 29, or LE₅AYG, and
(ii) a trivalent metal ion to a subject in need of such treatment.

81. A method according to claim 73, in which the non-amidated gastrin is Ggly₁₇.


SEQUENCE LISTING (19 sequences)

SEQ ID NO: 1 - protein, 80 aa, Homo sapiens
SWKPRSQQPDAPLGTGANRDLELPWLEQQGPASHHRRQLGPQGPPHLVADPSKKQGPWLEEEEEAYGWMDFGRRSAEDEN

SEQ ID NO: 2 - protein, 7 aa, Homo sapiens
EEEEEAY

SEQ ID NO: 3 - protein, 7 aa, Homo sapiens
LEEEEEA

SEQ ID NO: 4 - protein, 18 aa, Homo sapiens
  MISC_FEATURE (1)..(1): pyroglutamate
XGPWLEEEEEAYGWMDFG

SEQ ID NO: 5 - protein, 17 aa, Homo sapiens
  MOD_RES (17)..(17): AMIDATION
  MISC_FEATURE (1)..(1): pyroglutamate
XGPWLEEEEEAYGWMDF

SEQ ID NO: 6 - protein, 4 aa, Homo sapiens
  MISC_FEATURE (1)..(1): pyroglutamate
XGPW

SEQ ID NO: 7 - protein, 11 aa, Homo sapiens
  MISC_FEATURE (1)..(1): pyroglutamate
XGPWLEEEEEA

SEQ ID NO: 8 - protein, 7 aa, Homo sapiens
YGWMDFG

SEQ ID NO: 9 - protein, 14 aa, Homo sapiens
LEEEEEAYGWMDFG

SEQ ID NO: 10 - protein, 18 aa, Artificial Sequence
  Based on Human Ggly
  MISC_FEATURE (1)..(1): pyroglutamate
XGPWLAEEEEAYGWMDFG

SEQ ID NO: 11 - protein, 18 aa, Artificial Sequence
  Based on Human Ggly
  MISC_FEATURE (1)..(1): pyroglutamate
XGPWLEAEEEAYGWMDFG

SEQ ID NO: 12 - protein, 18 aa, Artificial Sequence
  Based on Human Ggly
  MISC_FEATURE (1)..(1): pyroglutamate
XGPWLEEAAAAYGWMDFG

SEQ ID NO: 13 - protein, 18 aa, Artificial Sequence
  Based on Human Ggly
  MISC_FEATURE (1)..(1): pyroglutamate
XGPWLAAAAAAYGWMDFG

SEQ ID NO: 14 - protein, 17 aa, Artificial Sequence
  Based on Human Gamide
  MISC_FEATURE (1)..(1): pyroglutamate
  MOD_RES (17)..(17): AMIDATION
XGPWLEAEEEAYGWMDF

SEQ ID NO: 15 - protein, 17 aa, Artificial Sequence
  Based on Human Ggly
  MISC_FEATURE (1)..(1): pyroglutamate
  MOD_RES (17)..(17): AMIDATION
XGPWLEEAAEAYGWMDF

SEQ ID NO: 16 - protein, 9 aa, Artificial Sequence
  Based on Human Ggly
LEEEEEAYG

SEQ ID NO: 17 - protein, 6 aa, Artificial Sequence
  Based on Human Ggly
LEEEEE

SEQ ID NO: 18 - protein, 6 aa, Artificial Sequence
  Based on Human Ggly
EEEEEA

SEQ ID NO: 19 - protein, 5 aa, Artificial Sequence
  Based on Human Ggly
EEEEE